(19) United States
(12) Patent Application Publication
(10) Pub. No.: US 2024/0000553 A1
(43) Pub. Date: Jan. 4, 2024

(54) METHOD AND SYSTEM FOR FABRICATING A DENTAL APPLIANCE

(71) Applicant: CARESTREAM DENTAL LLC, Atlanta, GA (US)

(72) Inventors: Shoupu CHEN, Rochester, NY (US); Victor C. WONG, Pittsford, NY (US); Jean-Marc INGLESE, Bussy-Saint-Georges (FR); Edward R. SHELLARD, Atlanta, GA (US); Delphine REYNARD, Montreuil (FR); Xavier RIPOCHE, Marne La Vallee (FR); Sabrina CAPRON-RICHARD, Noisiel (FR); Sebastien HENRY, Arcueil (FR)

(21) Appl. No.: 18/039,616

(22) PCT Filed: Dec. 2, 2021
(86) PCT No.: PCT/US21/61594

§ 371 Date: May 31, 2023

Related U.S. Application Data
(60) Provisional application No. 63/120,461, filed on Dec. 2, 2020.

(51) Int. Cl. A61C 13/34; A61C 7/00; A61C 7/08; A61C 9/00; B33Y 80/00; B33Y 50/00; B33Y 10/00; B33Y 30/00

(52) U.S. Cl. CPC A61C 13/34; A61C 7/002; A61C 7/08; A61C 9/0053; B33Y 80/00; B33Y 50/00; B33Y 10/00; B33Y 30/00; A61B 6/14

(57) ABSTRACT

A method and system for fabricating a dental appliance for orthodontic treatment. The method comprises the steps of obtaining three-dimensional dental data from a patient scan, locating initial tooth positions, generating optimal arch forms, and determining a digital model for fabricating an orthodontic aligner with additive device. The system comprises a scanning apparatus to acquire three-dimensional dental data, and a computer apparatus programmed with instructions for generating optimal arch forms, and determining a digital model for fabricating an orthodontic aligner with additive device.

### FIELD OF THE INVENTION

The present invention relates to a method and a system for fabricating a dental appliance, and more particularly to a method and a system for fabricating a dental appliance for orthodontic treatment by using image processing in biometrics analysis of x-ray computed tomography, optical, optical coherence tomography (OCT), or other dental imaging modalities generating 3D dental images.

### BACKGROUND OF THE INVENTION

Orthodontics deals with the diagnosis, prevention and correction of malpositioned teeth and jaws. Orthodontic treatment is to correct dental displacement of a patient. Usually, dental braces are applied to the patient's dental arch with tooth displacement. Until recent years, vacuum-formed aligners are more widely used. Transparent and smooth vacuum-formed aligners provide improved wearing experience and some other benefits during the treatment. Vacuum-formed aligners move teeth incrementally to reduce mild overcrowding and improve mild irregularities. At the beginning of the treatment process, a digitized three dimensions model of the patient's jaw with each tooth in an original natural position is acquired. Based on the initial teeth digital model, a specialized computer program with an operator's input analyzes teeth positions to be corrected, and then makes orthodontic treatment plan to move teeth to a final arrangement, which is generated based on algorithms built in the program with further operator's input according to practice experience or patient's need or preference. Based on both the initial teeth digital model and planned final arrangement (the final teeth digital model), a series of intermediate teeth digital models are defined in the process of treatment planning and a plurality of aligners are fabricated accordingly. The patient's teeth are repositioned from their initial tooth arrangement to the final tooth arrangement by placing a series of incremental position and orientation adjustment aligners over the patient's teeth during the orthodontic treatment.

Various imaging modalities can be used to generate 3D image data, for calculation in various types of appliance fabrication systems. The schematic diagram of FIG. 1 shows an exemplary fabrication system 260 for forming an orthodontic appliance. Image data from an imaging apparatus 270, such as a CBCT, an optical and an optical coherence tomography (OCT) imaging system, can be provided as a data file or as streamed data over a wired or wireless network 282, such as an ethernet network with internet connectivity. The network 282 can be used to transfer this 3D image data to a memory 284 or storage on a networked server or workstation 280. Clinical indications and related parameters for the patient can be entered or otherwise associated with the 3D data through workstation 280. An automated or partially automated process can then execute at workstation 280, generating an appliance design by forming a print file 288 or other data structure supportive of appliance fabrication. Print file 288 or other fabrication instructions can go to a fabrication system 290, which is also in signal communication with network 282.

The fabrication process of FIG. 1 can be highly automated or partially automated, or may be a manual fabrication process that uses the vector data provided by the system. The fabrication system 290 can include a networked 3D printer used to generate teeth arrangements and other appliances for fabricating a physical aligner. The operator or practitioner at workstation 280 can provide various control functions and commands for 3D printer operation using the biometric analysis tools described herein. Other types of fabrication system 290 may require additional setup or control and may require more extensive operator interaction or practitioner input in order to apply biometric analysis results Imaging modalities providing 3D image data for calculation in the appliance fabrication systems can include CBCT, optical and OCT imaging systems.

U.S. Pat. No. 6,879,712, entitled “System and method of digitally modeling craniofacial features for the purposes of diagnosis and treatment predictions” to Tuncay et al., appears to disclose a method of generating a computer model of craniofacial features. The three-dimensional facial features data are acquired using laser scanning and digital photographs; dental features are acquired by physically modeling the teeth. The models are laser scanned. Skeletal features are then obtained from radiographs. The data are combined into a single computer model that can be manipulated and viewed in three dimensions. The model also has the ability for animation between the current modeled craniofacial features and theoretical craniofacial features.

U.S. Pat. No. 6,250,918, entitled “Method and apparatus for simulating tooth movement for an orthodontic patient” to Sachdeva et al., seems to disclose a method of determining a 3-D direct path of movement from a 3-D digital model of an actual orthodontic structure and a 3-D model of a desired orthodontic structure. This method simulates tooth movement based on each tooth's corresponding three-dimensional direct path using laser scanned crown and markers on the tooth surface for scaling. There is no true whole tooth 3-D data available using the method described.

PCT application PCT/US2018/048070 entitled “Method of optimization in orthodontic applications” to Shoupu et al., appears to disclose a method of generating metrics indicative of tooth positioning along a dental arch of a patient, from tooth 3-D data acquired from the patient.

Transparent and smooth vacuum-formed aligners provide improved wearing experience and some other benefits during the treatment, but sometimes they may not suitable for complex orthodontic treatment as not providing enough required force to move some individual tooth.

U.S. Pat. No. 9,161,823 B2, entitled “Orthodontic systems and methods including parametric attachments” to John Morton et al., appears to describe a method of receiving a digital model of the patient's tooth and determining a desired force system for eliciting the selected tooth movement. In the method, a patient-customized attachment which is configured to engage an orthodontic appliance when worn by the patient, and to apply a repositioning force to the tooth is designed. The attachment is comprised of parameters having values selected based on the digital model, selected force system, and patient-specific characteristics.

U.S. Patent Application No. 2006/0199140 seems to teach a method for treating a patient's teeth that includes determining an initial configuration of the patient's teeth, determining a final configuration of the patient's teeth, designing a movement path for at least one of the patient's teeth from the initial configuration to the final configuration, dividing the movement path into a plurality of successive treatment steps, and producing two or more or dental aligners of substantially identical shape for at least one of the treatment steps in accordance with the target configuration.

EP2932935B1 appears to disclose methods of making aligners that includes features that enhance the performance and comfort of the aligner, including features for preventing damage or wear on the appliance; said aligners include fluid-permeable aligners, wrinkled aligners, modular aligners, aligners of varying thickness, aligners of varying stiffness, snap-on aligners, texture aligners, aligners having multiple layers, and lateral correction aligners. U.S. Pat. No. 6,293,790, discloses a system of steel dental pliers useful for modifying suck-down polymetric aligners.

EP1682029B1 seems to teach a method for producing orthodontic aligners to accommodate aligner auxiliaries by forming openings in the aligner to receive said auxiliaries.

U.S. Pat. No. 8,439,672 appears to describe a method and system for establishing an initial position of a tooth, a target position of the tooth in a treatment plan, calculating a movement vector associated with the tooth movement from the initial position to the target position, determining a plurality of components corresponding to the movement vector, and determining a corresponding one or more positions of a respective one or more attachment devices relative to a surface plane of the tooth such that the one or more attachment devise engages with a dental appliance.

Thus, there would be particular value in development of a method and a system for fabricating a dental appliance for orthodontic treatment.

### SUMMARY OF THE INVENTION

It is an object of the present disclosure to address the need for improved ways for fabricating a dental appliance for orthodontic treatment. With this object in mind, the present disclosure provides a method for generating arch forms with the final teeth digital model based on patient dentition as guidance tools for correcting orthodontic conditions.

According to an aspect of the present disclosure, there is provided a method for fabricating a dental appliance for orthodontic treatment, at least partially performed by a computer, comprising (a) obtaining three-dimensional dental data from a patient's scan, including at least one dental arch of the patient; (b) locating initial tooth positions along the dental arch from the three-dimensional dental data; (c) generating optimal arch forms for the patient's dental arch to acquire incremental positions and corresponding movement vectors for individual tooth in the dental arch; (d) determining a digital model for fabricating an orthodontic aligner with additive device based on the incremental positions and movement vectors; (e) displaying, storing, or transmitting the determined digital models.

According to some embodiments of the present disclosure, the method further comprises: (a) producing a physical model according to the determined digital model, using a 3D printer; and (b) fabricating a physical aligner with the additive device using the physical model.

According to some embodiments of the present disclosure, the three-dimensional dental data is three-dimensional volume representing dental anatomy of a patient acquired using a cone beam computed tomography system.

According to some embodiments of the present disclosure, the three-dimensional dental data is three-dimensional surface representing a tooth or teeth of a patient acquired using an intraoral optical scanner.

According to some embodiments of the present disclosure, the three-dimensional dental data is acquired using an optical coherence tomography (OCT) system.

According to some embodiments of the present disclosure, generating optimal arch forms for the patient's dental arch comprises steps of: (a) selecting a first positional digital data for one or more teeth from the located initial tooth positions along the dental arch from the three-dimensional dental data; (b) generating second positional digital data for the one or more teeth according to a desired dental arch form for the patient; (c) calculating displacement data for one or more teeth according to the first positional and second positional digital data; and (d) calculating an intermediate displacement for incremental positions and corresponding movement vectors for the one or more teeth.

According to some embodiments of the present disclosure, generating optimal arch forms for the patient's dental arch comprises steps of: (a) selecting a first positional digital data for one or more teeth from the located initial tooth positions along the dental arch from the three-dimensional dental data; (b) generating second positional digital data for the one or more teeth according to a desired dental arch form for the patient; (c) calculating a first displacement data for one or more teeth according to the first positional and second positional digital data; (d) detecting teeth collision values based on the first displacement data; (e) calculating a second displacement data for one or more teeth based on the detected teeth collision values; (f) combining the first displacement data and second displacement data; (g) calculating an intermediate displacement for incremental positions and corresponding movement vectors for the one or more teeth; and (h) reporting the intermediate displacement for repositioning one tooth or more teeth of the dental arch.

According to yet another aspect of the present disclosure, there is provided an apparatus system for dental orthodontic treatment, comprises: (a) a scanning apparatus configured to acquire three-dimensional dental data from a patient's scan; (b) a computer apparatus programmed with instructions for: (i) locating initial tooth positions along the dental arch from the three-dimensional dental data; (ii) generating optimal arch forms for the patient's dental arch to acquire incremental positions and corresponding movement vectors for individual tooth in the dental arch; (iii) determining a digital model for fabricating an orthodontic aligner with additive device based on the incremental positions and movement vectors; (iv) displaying, storing, or transmitting the determined digital models.

According to some embodiments of the present disclosure, the scanning apparatus is a cone beam computed tomography (CBCT) system, an intraoral optical scanner, an optical coherence tomography (OCT) system, or any combination of the foregoing.

According to some embodiments of the present disclosure, the apparatus system further comprises: (a) a 3D printer for producing a physical model according to the determined digital model, wherein the 3D printer is in signal communication with the computer apparatus; (b) an apparatus for fabricating a physical aligner with additive device using the physical model.

Embodiments of the present disclosure, in a synergistic manner, integrate skills of a human operator of the system with computer capabilities for feature identification. This takes advantage of human skills of creativity, use of heuristics, flexibility, and judgment, and combines these with computer advantages, such as speed of computation, capability for exhaustive and accurate processing, and reporting and data access capabilities.

These and other aspects, objects, features and advantages of the present disclosure will be more clearly understood and appreciated from a review of the following detailed description of the preferred embodiments and appended claims, and by reference to the accompanying drawings.

### BRIEF DESCRIPTION OF THE DRAWINGS

The foregoing and other objects, features, and advantages of the invention will be apparent from the following more particular description of the embodiments of the disclosure, as illustrated in the accompanying drawings. The elements of the drawings are not necessarily to scale relative to each other.

FIG. 1 is a schematic diagram showing an exemplary fabrication system for forming an orthodontic appliance.

FIG. 2A lists example parameters as numerical values and their interpretation.

FIGS. 2B, 2C and 2D list, for a particular patient, example parameters as numerical values and their interpretation with respect to maxillofacial asymmetry, based on exemplary total maxillofacial asymmetry parameters according to exemplary embodiments of this application.

FIG. 3A shows exemplary tabulated results for a particular example with bite analysis and arches angle characteristics.

FIG. 3B shows exemplary tabulated results for a particular example for torque of upper and lower incisors.

FIG. 3C shows exemplary tabulated results for another example with assessment of biretrusion or biprotrusion.

FIG. 3D shows an exemplary summary listing of results for cephalometric analysis of a particular patient.

FIG. 3E shows a detailed listing for one of the conditions listed in FIG. 6.

FIG. 4 shows a system display with a recommendation message based on analysis results.

FIG. 5 shows a system display with a graphical depiction to aid analysis results.

FIG. 6 shows an exemplary report for asymmetry according to an embodiment of the present disclosure.

FIG. 7 is a logic flow diagram that shows the logic processing mechanisms and data that can be used for providing assessment and guidance to support orthodontic applications.

FIG. 8 shows an image of a typical patient condition, arch left-rotation.

FIG. 9 shows a 3-D mapping of tooth inertia centers along an arch.

FIG. 10 shows a plot of tooth inertia centers arranged along a piecewise linear curve that connects the inertia centers, in their original positions, projected to the 2D x-y plane.

FIG. 11 shows, for the inertia centers in FIG. 10, the relative position of an optimized smooth polycurve.

FIG. 12 shows needed motion of the teeth from their original positions to desired positions based upon the computed optimization as displayed and reported.

FIG. 13 shows a graph with tooth displacement vectors indicated, based on the above computation.

FIG. 14 shows a graph with vector displacement decomposed into tangential and normal directions.

FIGS. 15 and 16 show adjustments for a multi-stage tooth arch form optimization strategy adopted according to the present disclosure.

FIG. 17 shows a computer-generated listing of movement vectors V for each of 14 teeth of a dental arch for an orthodontic patient.

FIG. 18A shows the distribution of teeth in an initial, uncorrected arch for an exemplary orthodontic patient.

FIG. 18B shows the digitally corrected arch of FIG. 18A following treatment recommendations provided by the system of the present disclosure.

FIG. 18C shows initial and optimized arches overlaid, with the original shown in outline, in a 3D view.

FIG. 18D shows another example with the initial arch in outline, overlaid on the optimized arch, in a 2D axial view.

FIG. 19 shows an operator interface display for controlling the processing to provide orthodontic data and for display of processing results.

FIG. 20 is a logic flow diagram showing a logic processing mechanism and data that can be used for providing assessment and guidance to support orthodontic applications according to an embodiment of the present invention.

FIG. 21A is a diagram showing a distribution of teeth in an initial uncorrected dental arch from an exemplary orthodontic patient.

FIG. 21B is a diagram showing a digitally optimized arch shape with teeth collision.

FIG. 21C is a diagram showing a digitally optimized arch shape without teeth collision.

FIG. 21D is a diagram indicating a moving direction and a moving distance of a tooth digital model according to an embodiment of the present invention.

FIG. 22 is a logic flow diagram showing process to calculate a moving direction and a moving distance of a tooth digital model with collisions according to an embodiment of the present invention.

FIG. 23 is a logic flow diagram that shows a sequence for applying results of the orthodontic optimization to the task of appliance design and fabrication.

FIG. 24 is a diagram showing an example of tooth repositioning.

FIG. 25 is a diagram showing an example of a conventional aligner fabricated based on the digital model for an incremental position (or orientation) adjustment of the teeth.

FIG. 26 is a diagram showing an example of a conventional aligner on the teeth to be repositioned.

FIG. 27 is a diagram showing an example of a modified aligner fabricated based on the digital model determined according to an embodiment of the present invention.

FIG. 28 depicts part of a digital model corresponding to the model, with two negative ellipsoids added.

FIG. 29 is a diagram showing an example of a modified aligner fabricated based on the digital model determined in the present invention on the teeth to be repositioned.

FIG. 30 is a diagram showing an operating principle of a modified aligner according to an embodiment of the present invention.

### DETAILED DESCRIPTION OF THE INVENTION

In the following detailed description of embodiments of the present disclosure, reference is made to the drawings in which the same reference numerals are assigned to identical elements in successive figures. It should be noted that these figures are provided to illustrate overall functions and relationships according to embodiments of the present invention and are not provided with intent to represent actual size or scale.

Where they are used, the terms “first”, “second”, “third”, and so on, do not necessarily denote any ordinal or priority relation, but may be used for more clearly distinguishing one element or time interval from another.

In the context of the present disclosure, the term “image” refers to multi-dimensional image data that is composed of discrete image elements. For 2-D images, the discrete image elements are picture elements, or pixels. For 3-D images, the discrete image elements are volume image elements, or voxels. The term “volume image” is considered to be synonymous with the term “3-D image”. In the context of the present disclosure, the term “code value” refers to the value that is associated with each 2-D image pixel or, correspondingly, each volume image data element or voxel in the reconstructed 3-D volume image. The code values for computed tomography (CT) or cone-beam computed tomography (CBCT) images are often, but not always, expressed in Hounsfield units that provide information on the attenuation coefficient of each voxel.

In the context of the present disclosure, the term “geometric primitive” relates to an open or closed shape such as a rectangle, circle, line, traced curve, or traced pattern. The terms “landmark” and “anatomical feature” are considered to be equivalent and refer to specific features of patient anatomy as displayed.

In the context of the present disclosure, the terms “viewer”, “operator”, and “user” are considered to be equivalent and refer to the viewing practitioner or other person who views and manipulates an image, such as a dental image, on a display monitor. An “operator instruction” or “viewer instruction” is obtained from explicit commands entered by the viewer, such as using a computer mouse or touch screen or keyboard entry.

The term “highlighting” for a displayed feature has its conventional meaning as is understood to those skilled in the information and image display arts. In general, highlighting uses some form of localized display enhancement to attract the attention of the viewer. Highlighting a portion of an image, such as an individual organ, bone, or structure, or a path from one chamber to the next, for example, can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at higher sharpness or contrast.

In the context of the present disclosure, the descriptive term “derived parameters” relates to values calculated from processing of acquired or entered data values. Derived parameters may be a scalar, a point, a line, a volume, a vector, a plane, a curve, an angular value, an image, a closed contour, an area, a length, a matrix, a tensor, or a mathematical expression.

The term “set”, as used herein, refers to a non-empty set, as the concept of a collection of elements or members of a set is widely understood in elementary mathematics. The term “subset”, unless otherwise explicitly stated, is used herein to refer to a non-empty proper subset, that is, to a subset of the larger set, having one or more members. For a set S, a subset may comprise the complete set S. A “proper subset” of set S, however, is strictly contained in set S and excludes at least one member of set S. Alternately, more formally stated, as the term is used in the present disclosure, a subset B can be considered to be a proper subset of set S if (i) subset B is non-empty and (ii) if intersection B ∩S is also non-empty and subset B further contains only elements that are in set S and has a cardinality that is less than that of set S.

In the context of the present disclosure, a “plan view” or “2-D view” is a 2-dimensional (2-D) representation or projection of a 3-dimensional (3-D) object from the position of a horizontal plane through the object. This term is synonymous with the term “image slice” that is conventionally used to describe displaying a 2-D planar representation from within 3-D volume image data from a particular perspective. 2-D views of the 3-D volume data are considered to be substantially orthogonal if the corresponding planes at which the views are taken are disposed at 90 (+/−10) degrees from each other, or at an integer multiple n of degrees from each other (n*90 degrees, +/−10 degrees).

In the context of the present disclosure, the general term “dentition element” relates to teeth, prosthetic devices such as dentures and implants, and supporting structures for teeth and associated prosthetic device, including jaws.

The terms “poly-curve” and “polycurve” are equivalent and refer to a curve defined according to a polynomial.

The present disclosure relates to digital image processing and computer vision technologies, which is understood to mean technologies that digitally process data from a digital image to recognize and thereby assign useful meaning to human-understandable objects, attributes or conditions, and then to utilize the results obtained in further processing of the digital image.

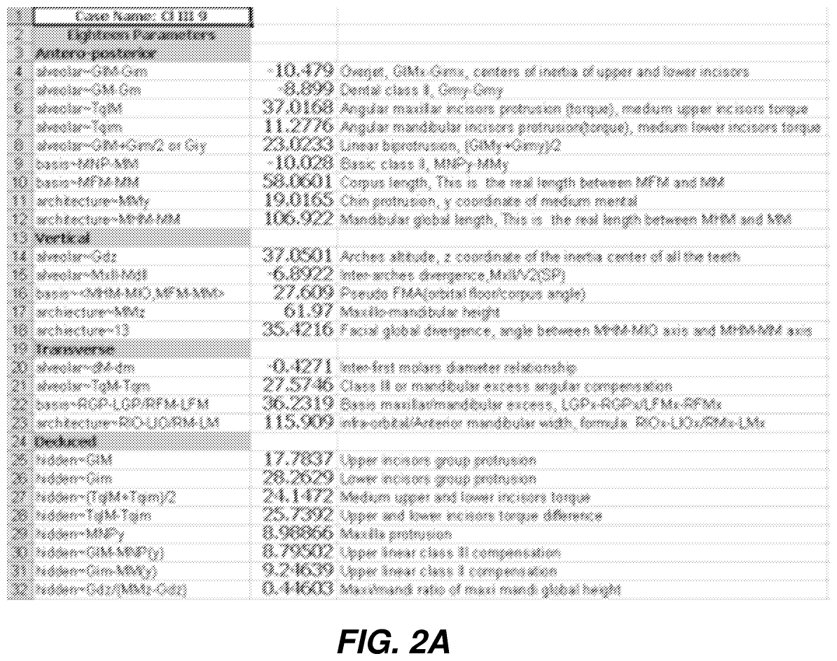

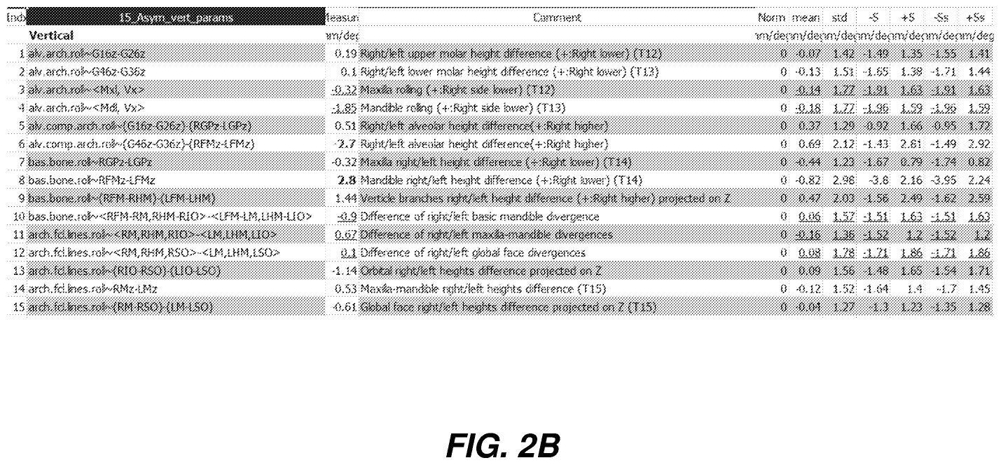

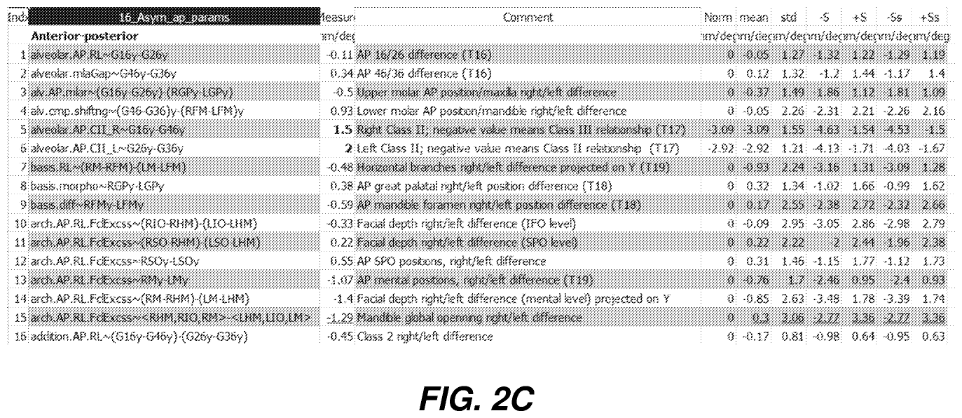

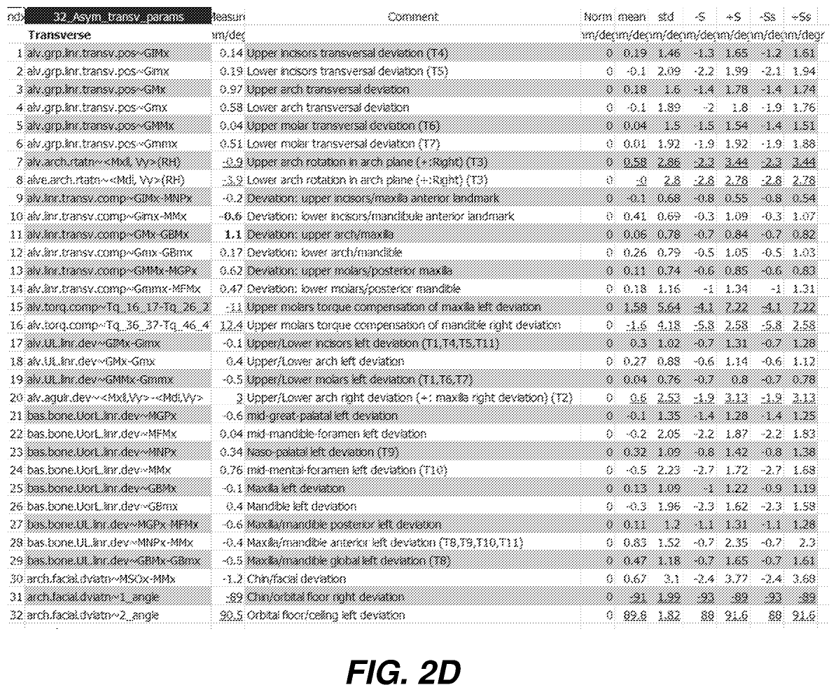

The present invention provides a method and a system for fabricating a dental appliance for orthodontic treatment, where an improved teeth digital model for fabricating physical aligners is provided according to incremental positions (or orientation) and corresponding movement vectors that may include translational and angular elements (for orientation correction) for individual teeth within the dental arch; translational elements are acquired from optimal arch forms for the patient's dental arch. Exemplary angular motion elements are angles related to the Euler angles of a tooth with respect to a local orthogonal coordinate system centered at the inertia center of said tooth. An exemplary angular variable, TqIM, that measures the upper incisor torque, is illustrated in FIG. 2A. An angular compensation for a tooth's torque may be needed for the final comprehensive teeth model for orthodontic appliance fabrication. The fabricating methods and system of the present invention includes determination of a digital teeth model for the fabrication of an orthodontic aligner with additive device based on the incremental positions and movement vectors. Comparing with most of the conventional aligner, the new aligner introduced in the present invention provides more specialized forces to the individual teeth, to improve the efficiency in desired orthodontic treatment.

An embodiment of the present disclosure uses measurements of relative positions of teeth and related anatomy from either CBCT, optical scanning, optical coherence tomography (OCT), or any possible combination of all as input to an analysis processor or engine for maxillofacial/dental biometrics. The biometrics analysis processor, using artificial intelligence (AI) algorithms and related machine-learning approaches, generates diagnostic orthodontic information that can be useful for patient assessment and ongoing treatment. Using the generated AI output data and analysis from the biometrics analysis processor, an AI inverse operation then generates and displays quantitative data to support corrective orthodontics.

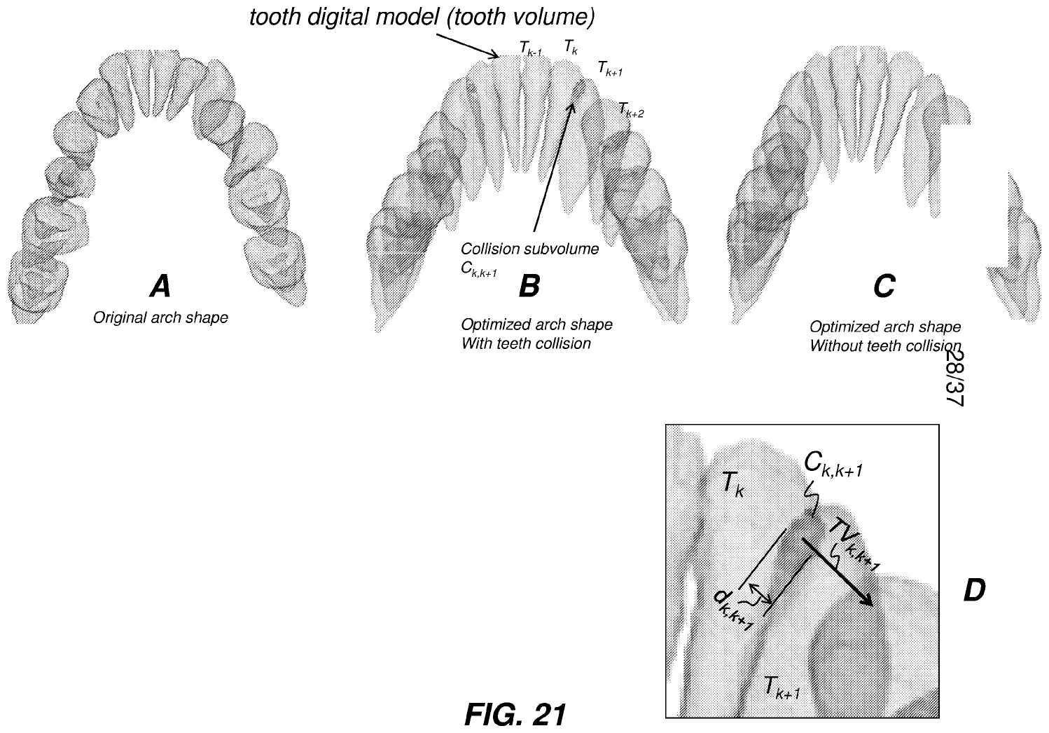

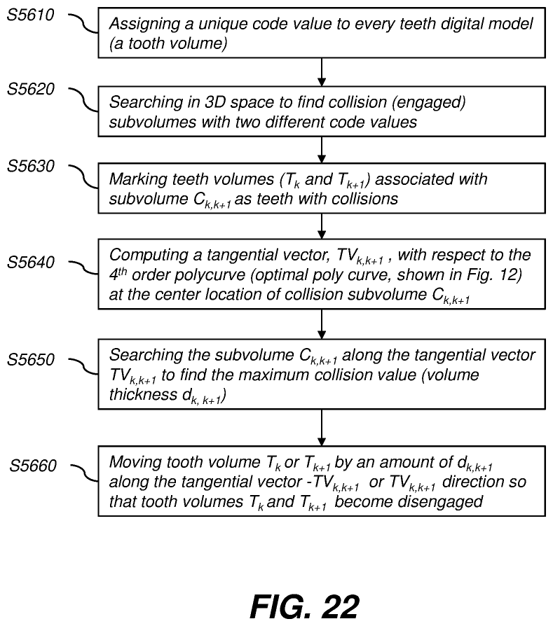

According to an embodiment of the present disclosure, the described method provides an automated solution for defining an optimal arch form based on the teeth positions of the individual patient prior to orthodontic treatment. The optimized arch form can be represented by a teeth model such as the one displayed by FIG. 21C.

Guidance can also be provided for use of dental appliances, including design, use, placement arrangements. To support deployment of orthodontic appliances, an embodiment of the present disclosure provides guidance for a multi-step process toward achieving an optimal arch form. For the individual patient, the method computes a set having multiple recommended motion vectors that can be used to direct tooth repositioning. According to an alternate embodiment, one or more of the appropriate dental appliances can be fabricated in whole or in part using a 3D printer, if feasible; an appropriate appliance can alternately be assembled using an arrangement of standard brackets and braces.

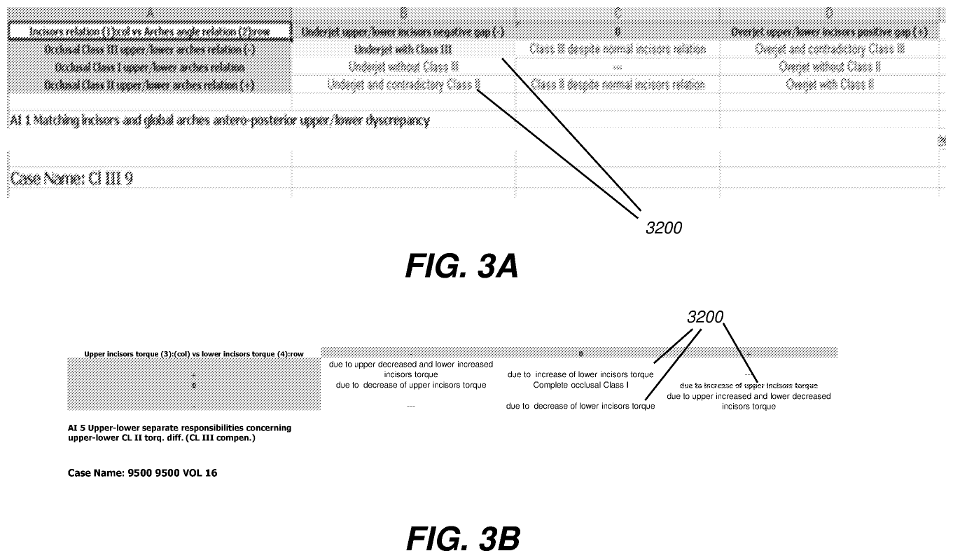

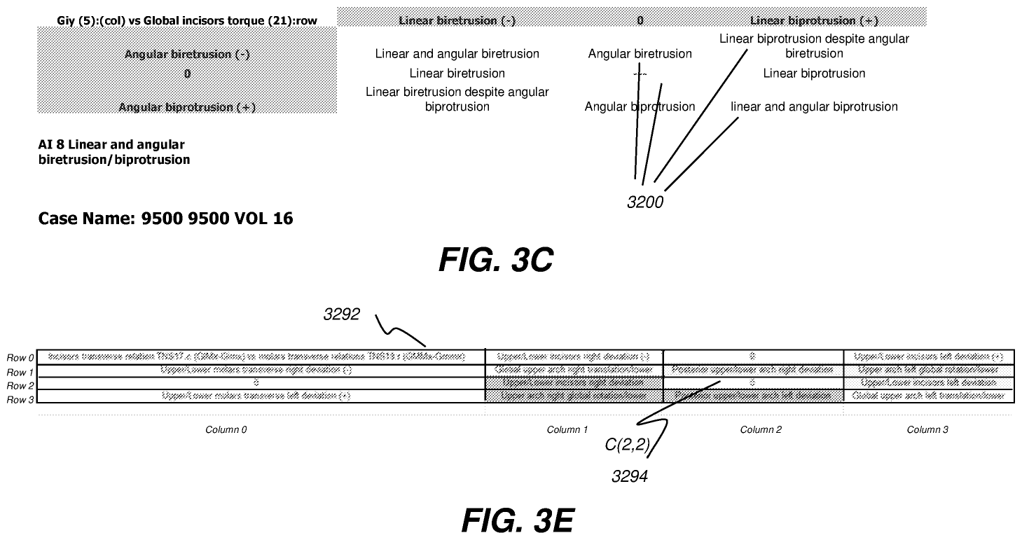

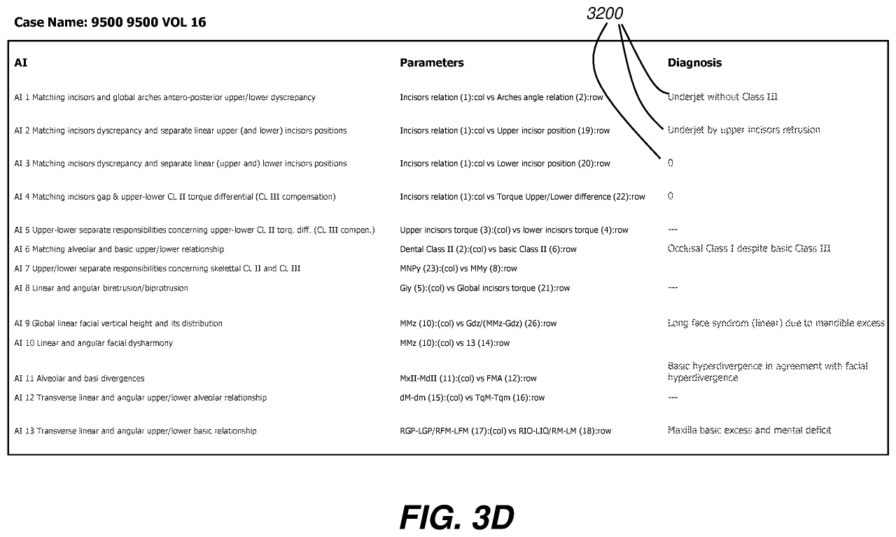

Now we will use data from CBCT reconstruction as an example to describe dental/maxillofacial abnormalities in detail, but a person skilled in the art should know that the similar abnormalities can also be observed from data of other imaging modalities, such as optical scanner, OCT scanner and the like. FIG. 2A lists, for a particular patient, example parameters as numerical values and their interpretation with respect mainly to malocclusion of teeth, based on the listing of 26 parameters given previously. FIGS. 2B, 2C and 2D list, for a particular patient, example parameters as numerical values and their interpretation with respect to maxillofacial asymmetry, based on the listing of total 30 parameters given in an exemplary embodiment of this application. FIG. 3A shows exemplary tabulated results 3200 for a particular example with bite analysis and arches angle characteristics. In the example of FIG. 3A, the columns indicate an underjet, normal incisors relation, or overjet condition. Rows represent occlusal classes and arches angle conditions. As FIG. 3A shows, highlighting can be used to accentuate the display of information that indicates an abnormal condition or other condition of particular interest. For the particular patient in the FIG. 3A example, analysis indicates, as a result, an underjet condition with Class III bite characteristics. This result can be used to drive treatment planning, depending on severity and practitioner judgment.

FIG. 3B shows exemplary tabulated results 3200 for another example with analysis of torque for upper and lower incisors, using parameters 3 and 4 from the listing given previously.

FIG. 3C shows exemplary tabulated results 3200 for another example with assessment of biretrusion or biprotrusion using calculated parameters given earlier as parameters (5) and (21).

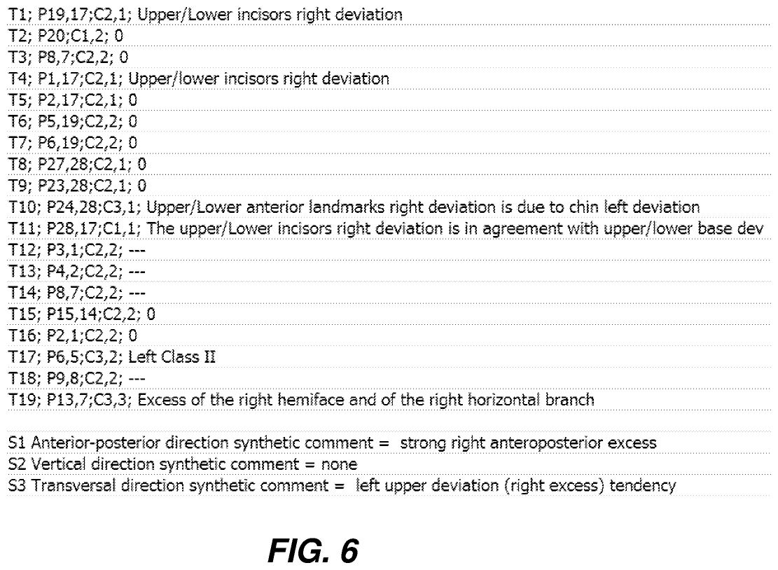

FIG. 32D shows an exemplary summary listing of results for cephalometric analysis of a particular patient. The listing that is shown refers to analysis indications taken relative to parameters 1-26 listed previously. In the particular example of FIG. 32D, there are 13 results for parameter comparisons using biometric parameters and dentition information derived as described herein. Additional or fewer results could be provided in practice. FIG. 3E shows a detailed listing for one of the conditions reported in a tabular listing with a table 3292 with cells 3294 as shown subsequently (FIG. 6).

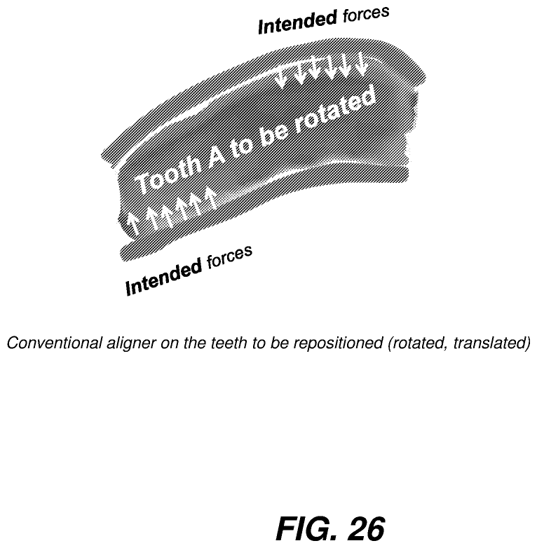

Results information from the biometry computation can be provided for the practitioner in various different formats. Tabular information such as that shown in FIGS. 2A-3E can be provided in file form, such as in a comma-separated value (CSV) form that is compatible for display and further calculation in tabular spreadsheet arrangement, or may be indicated in other forms, such as by providing a text message. A graphical display, such as that shown in FIG. 26, can alternately be provided as output, with particular results highlighted, such as by accentuating the intensity or color of the display for features where measured and calculated parameters show abnormal biometric relations, such as overjet, underjet, and other conditions.

The computed biometric parameters can be used in an analysis sequence in which related parameters are processed in combination, providing results that can be compared against statistical information gathered from a patient population. The comparison can then be used to indicate abnormal relationships between various features. This relationship information can help to show how different parameters affect each other in the case of a particular patient and can provide resultant information that is used to guide treatment planning.

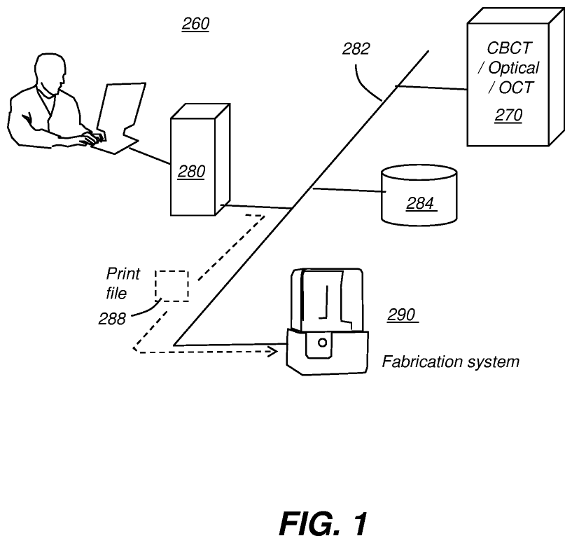

Referring back to FIG. 1, memory 132 can be used to store a statistical database of cephalometric information gathered from a population of patients. Various items of biometric data that provides dimensional information about teeth and related supporting structures, with added information on bite, occlusion, and interrelationships of parts of the head and mouth based on this data can be stored from the patient population and analyzed. The analysis results can themselves be stored, providing a database of predetermined values capable of yielding a significant amount of useful information for treatment of individual patients. According to an embodiment of the present disclosure, the parameter data listed in FIGS. 2A and 2B are computed and stored for each patient, and may be stored for a few hundred patients or for at least a statistically significant group of patients. The stored information includes information useful for determining ranges that are considered normal or abnormal and in need of correction. Then, in the case of an individual patient, comparison between biometric data from the patient and stored values calculated from the database can help to provide direction for an effective treatment plan.

As is well known to those skilled in the orthodontic and related arts, the relationships between various biometric parameters measured and calculated for various patients can be complex, so that multiple variables must be computed and compared in order to properly assess the need for corrective action. The analysis engine described in simple form with respect to FIGS. 3A-3E compares different pairs of parameters and provides a series of binary output values. In practice, however, more complex processing can be performed, taking into account the range of conditions and values that are seen in the patient population.

Highlighting particular measured or calculated biometric parameters and results provides useful data that can guide development of a treatment plan for the patient.

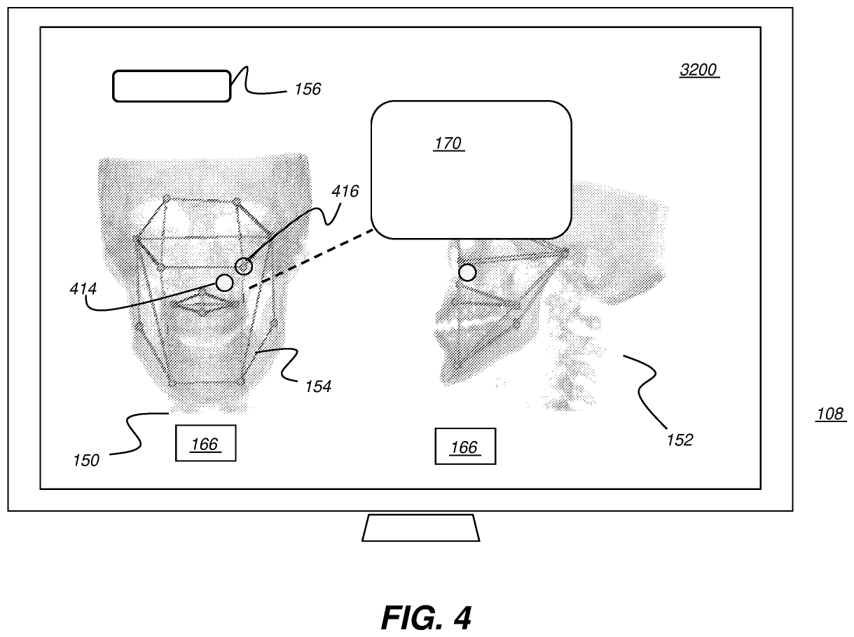

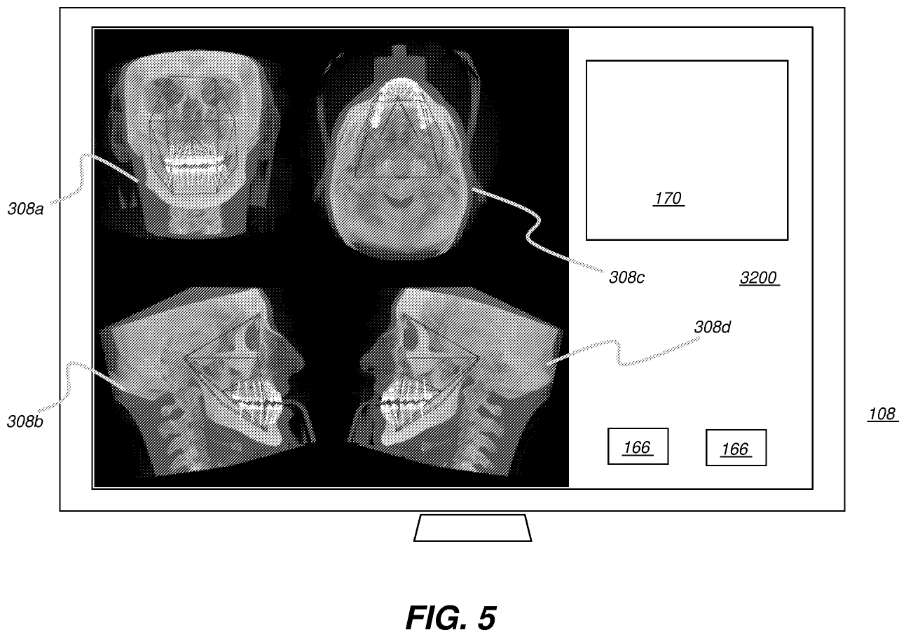

FIG. 4 shows a system display of results 3200 with a recommendation message 170 based on analysis results and highlighting features of the patient anatomy related to the recommendation. FIG. 5 shows a system display 108 with a graphical depiction of analysis results 3200. Annotated 3-D views (e.g., 308a-308d) are shown, arranged at different angles, along with recommendation message 170 and controls 166.

Certain exemplary method and/or apparatus embodiments according to the present disclosure can address the need for objective metrics and displayed data that can be used to help evaluate asymmetric facial/dental anatomic structure. Advantageously, exemplary method and/or apparatus embodiments present measured and analyzed results displayed in multiple formats suitable for assessment by the practitioner.

FIG. 6 shows an exemplary text report for maxillofacial asymmetry assessment according to an embodiment of the present disclosure. The report lists a set of assessment tables (T1-T19) available from the system, with cell entries (denoted by C with row and column indices, C(row, column)) providing maxillofacial/dental structural asymmetry property assessment comments organized based on the calculations related to relationships between obtained parameters, such as parameters P1-P15 in FIG. 2B. An exemplary assessment table 3292 is depicted in FIG. 3E, having four rows and four columns.

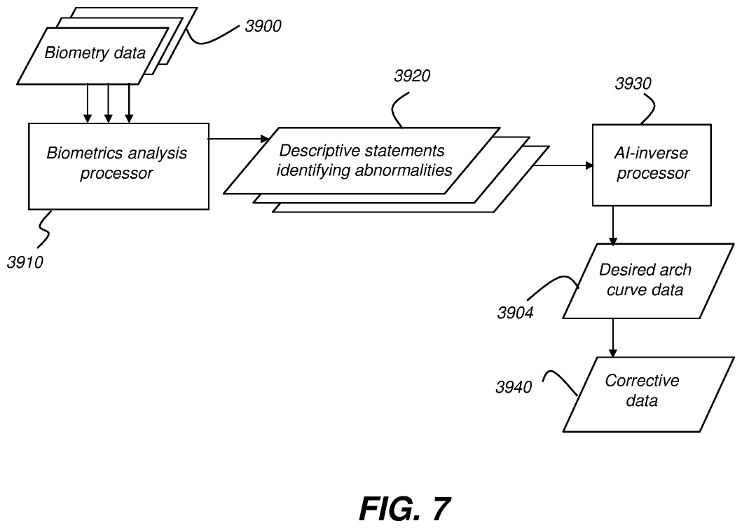

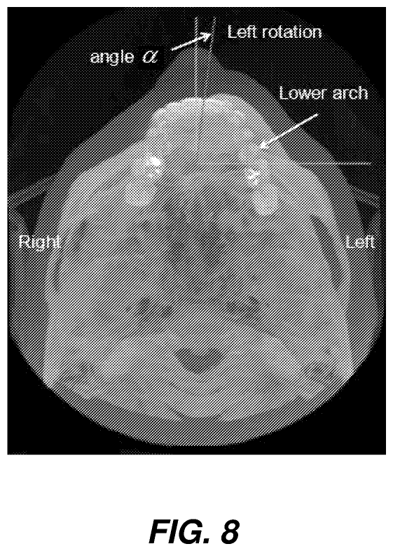

The flow diagram of FIG. 7 shows the logic processing mechanisms and data that can be used for providing assessment and guidance to support orthodontic applications. Biometry data 3900, obtained from CBCT, optical scanning, OCT scanning or other source, and population data is input to a biometrics analysis processor 3910. In response, biometrics analysis processor 3910, an artificial intelligence (AI) engine, performs calculations and generates descriptive statements 3920 identifying one or more dental/maxillofacial abnormalities. FIGS. 2A-3D illustrate examples of descriptive statements describing one or more dental/maxillofacial abnormalities.

Continuing with the FIG. 7 process, an AI-inverse processor 3930 provides a logic engine that generates recommended or desired arch curve data 3904 based on and generated from anatomy of the individual patient. Based on the descriptive statements 3920 and on the desired arch curve data 3904, AI-inverse processor 3930 then generates corresponding corrective data 3940 that can include the motion vectors needed for tooth repositioning, and related data for guidance in orthodontics. Now we will use data from CBCT reconstruction as an example to explain the process in detail, but a person skilled in the art should know that the method is also suitable for data from other imaging modalities, such as optical scanner, OCT scanner and the like.

AI-inverse processing can begin with arch form optimization. The example case shown in FIG. 8 shows an image of a typical patient condition, arch left-rotation by an angle α. A non-zero angle indicates tooth arch form asymmetry. The sequence performed for arch rotation correction provides an example of the activity of AI-inverse processor 3930 for this typical case. Sequence steps are outlined following:
1. The AI engine, biometry analysis processor 3910 of FIG. 7, analyzes biometric data, from the CBCT reconstruction for example, and generates descriptive statements indicative of the arch left-rotation condition shown in FIG. 8.
2. The AI-inverse engine, AI-inverse processor 3930, based on the descriptive statements and on recommended or desired results for arch characteristics, generates patient-specific, coherent and optimized geometric primitives and related parameters that indicate quantitative tooth movement values for the current stage of the treatment process.

For the example of FIG. 8:

(i) The AI engine detects an arch rotation that is a function ƒ(t) of tooth vector t representing the set {t₁, . . . t_{N}}, wherein N is the number of teeth in the arch. The positions (in an exemplary 2D space) of t can be corrected by the AI engine inverse operation by rearranging the teeth t to minimize the arch rotation in a systematic and automated manner:

min ƒ(t);

this expression is subject to an exemplary function g(t)=4^{th} order polycurve, which, in turn, leads to solving an over-determined system in an exemplary 2D space: X^{T}β=y
wherein X^{T} signifies a matrix that contains all the teeth's 0 to n^{th} order x positions in the exemplary 2D space. An exemplary value is n=4. Variable y signifies a vector {y₁, y_{N}} that contains all the teeth's 1^{st} order y positions in the exemplary 2D space; again, variable N is the number of teeth included in the arch. Where β signifies a vector {β₀ . . . βₙ} with the object function

$$\hat{β} = {arg min}_{β} S ( β ) .$$

The concept of corrective means is applicable to 3D space.
(ii) With the goal of achieving: min(ƒ(t)=α), the sequence can be as follows:
   (1) First, compute a tensor matrix I=I_{d}trace(C)−C; where C=ΣₖmₖpₖPₖ^{T} wherein d=2 or 3, for 2D or 3D calculation, correspondingly; and pₖ represents the (x,y,z) position vector of an element (a voxel of a tooth, for example).

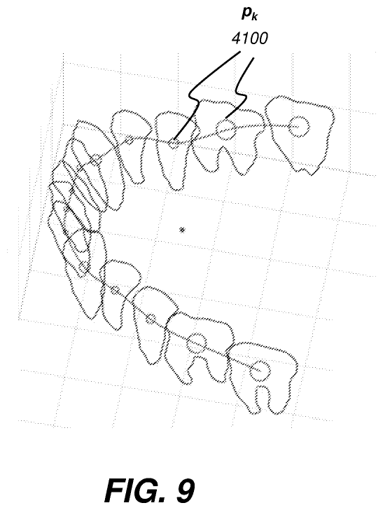

In the arch rotation case of FIG. 8, pₖ is the center of inertia of tooth k. k={1, 2, 3, . . . N}. Again, variable N is the number of teeth included in the arch. FIG. 9 shows, in a 3D perspective view, an exemplary representation of inertia centers pₖ 4100 along an arch. Embodiments of the present disclosure display the inertia centers in a 2D plane, showing recommended adjustments for inertia center positioning. The procedures for generating recommended adjustments can alternately be extended to correction in 3D space.
(2) Second, compute eigenvectors of tensor I in order to compute the arch rotation angle α;
(3) Third, compute the arch curve, such as g (t)=4^{th} potycurve using tooth inertia centers pₖ as input points.

It is noted that the set of inertia centers pₖ can be either augmented with additional input points or, alternately, reduced in size by removing outlier input points. Exemplary additional input points could be the original inertia centers with flipped sign (x direction); exemplary outlier points could be those whose coordinates show significant deviation from an ideal arch shape.

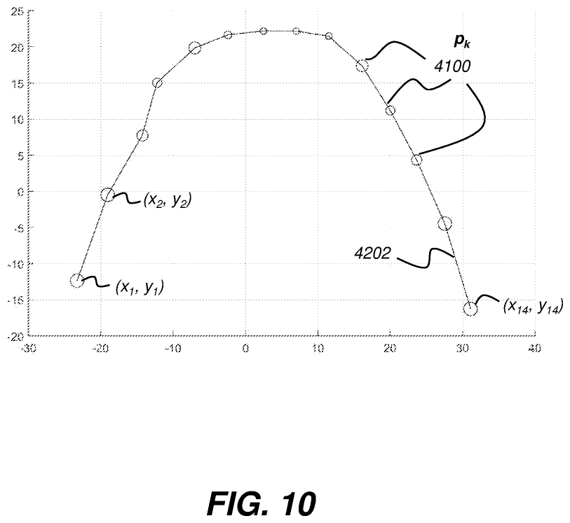

To simplify the problem, the 4^{th} order poly-curve (polynomial curve) \hat{β} can be computed in (x,y) space, that is, with variable d=2. FIG. 10 shows inertial centers 4100 plotted in x,y space (2D) along an initial, uncorrected piecewise linear arch curve 4202 with 14 teeth. A few of the inertial centers 4100 are labeled with exemplary coordinate designations {(x₁, y₁), (x₂, y₂), . . . (x₁₄, y₁₄)}.

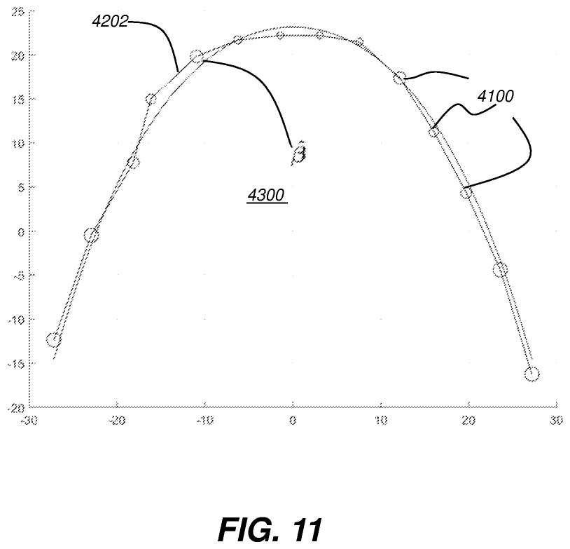

The poly-curve (polynomial curve) \hat{β} computation can be obtained by minimizing

$$S ( β ) = {\sum }_{i = 1}^{m} {\vert {y}_{i} - {\sum }_{j = 1}^{n} {x}_{ij} {β}_{j} \vert}^{2} ;$$

wherein yᵢ is an element of {y₁, . . . y_{N}}, xᵢⱼ is an element of matrix X^{T}. The above S(β) equation signifies an over-determined linear system that can be solved, for example, by using the well-known pseudo-inverse method familiar to individuals skilled in the art. Following this calculation, the tooth inertia centers 4100 shown in FIG. 10 can then be directly mapped. In one direct mapping method, the xₙ, value for each inertia center pₖ 4100 is fixed and the yₙ value adjusted to vertically shift the inertia center onto the polynomial curve \hat{β}. As shown in FIG. 11, the individual tooth inertia centers pₖ 4100 can be vertically moved by a slight amount in order to fit the polynomial curve, while holding the corresponding x values constant.

The original uncorrected centers can also be moved onto the polynomial curve \hat{β} by appropriately choosing one of the roots of the n^{th} order polynomial curve, holding the yₙ value as the fixed input and xₙ (the roots) as the output. The tensor matrix I can then be recomputed using the moved centers. Eigenvectors of the tensor can be recomputed and arch rotation angle α (FIG. 8) recalculated.

The processing described above can be repeated until angle α reaches a predetermined minimal value or zero (indicating a symmetric tooth arch form).

FIG. 11 shows the desired optimized arch curve \hat{β} 4300 overlaid for comparison with the original arch form before optimization, computed as described previously.

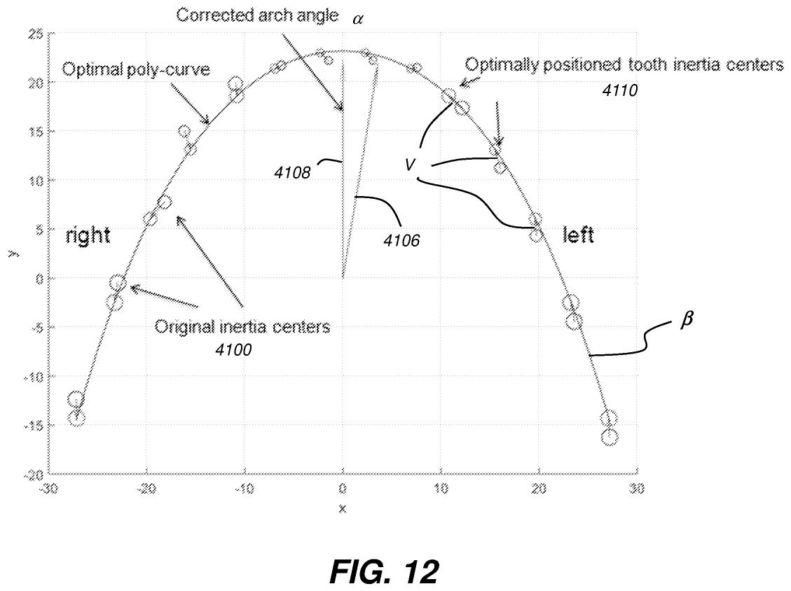

The recommended movement of the teeth from their original positions to desired positions based upon the computed optimization can be displayed and reported to the practitioner, as shown in FIG. 12.

In the reported data of FIG. 12, the original tooth inertia centers 4100 for the arch can be shown, such as highlighted in a particular color or with other suitable display treatment. Motion vectors V then show the desired movement from the original inertia center 4100 position to an optimized inertia center 4110. The original arch rotation is represented by a vector 4106. Corrected arch rotation angle α is represented by a vector 4108.

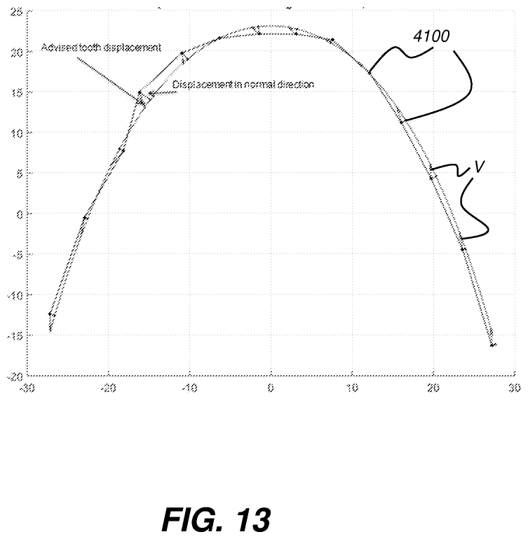

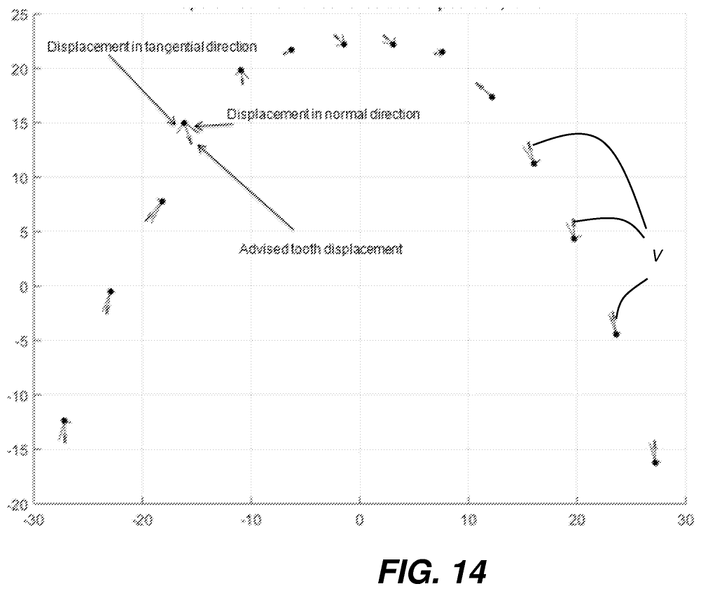

FIG. 13 shows a graph with tooth displacement vectors V indicated, based on the above computation. FIG. 14 shows a graph with vector displacement decomposed into mutually orthogonal tangential and normal component directions.

For the example shown in FIG. 8, corrective data showing teeth displacement vectors can help to serve as a guideline for orthodontic treatment. Exemplary optimized 4^{th} order polynomial curve intercept and coefficient parameters can be as follows:

\hat{β} [symbol] \hat{β}i;i={0, . . . 4}
23.174417248277855
−0.000000000000000
−0.037164341067977
0.000000000000000
−0.000018751015683

According to an embodiment of the present disclosure, the system provides the type of data presented in FIG. 14, in some form, to the practitioner to support an orthodontic treatment plan for a patient. For each tooth in the dental arch, a corresponding vector V is calculated based on the recommended or desired adjustment to tooth inertia center position.

### Multi-Stage Embodiment

An alternate embodiment of the present disclosure extends the logic for tooth position adjustment to correspond more closely to a multi-stage sequence for orthodontic treatment. This embodiment provides multiple iterations of the repositioning calculation and reporting process, tracking patient progress more closely to recommend the necessary adjustments at each stage.

The system of the present disclosure receives, at a time T₀, first positional data of the components (teeth) of the patient's dentition with the digital data extracted, through an AI-Engine, from at least one 3D digital volume acquisition modality applied to the dentition. The 3D volume could be acquired by using a CBCT scanner, an optical scanner, a laser scanner or an OCT scanner.

Automatically, through an AI-Engine inverse operation, the system of the present disclosure produces second positional digital data of the components (teeth) of the patient's dentition based on the first positional digital data of the dentition components with the second positional digital data highly optimized so that, at time T₁ after orthodontic treatment, the resultant arch form of the dentition components (teeth) is an improved fit for a number of aesthetic and functional requirements.

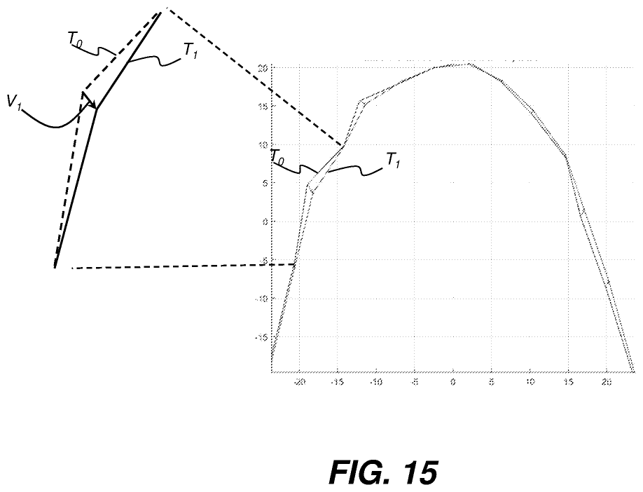

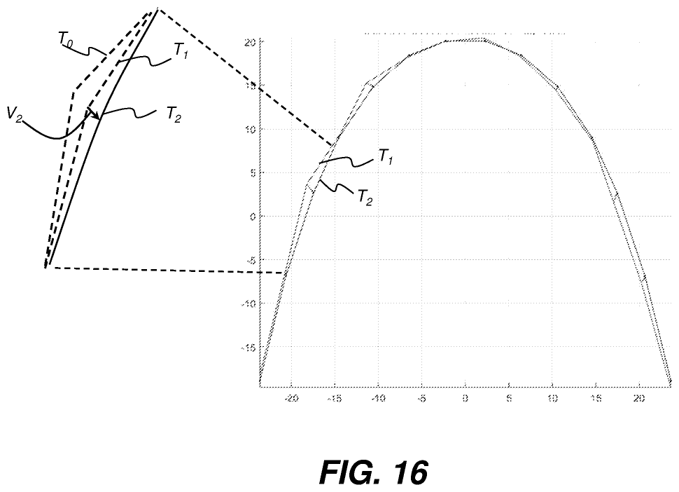

FIGS. 15 and 16 show a succession for arch improvement by moving teeth in stages using the iterative arch approximation calculations described herein. The graphical representation of FIG. 15 shows a first step executed using the AI-inverse operation, for dental arch shape from a 3D volume, acquired by using a CBCT scanner, an optical scanner, a laser scanner or an OCT scanner. The dashed line representation, in the partial enlarged section, shows a portion of the arch curve following extraction of the original patient data at time T₀.

In practice, at time T₀, a CBCT scan, intraoral optical scan, or an OCT scan are taken. 3D tooth models with roots can be generated from a CBCT scan; 3D crown models without roots can be generated from an optical scan or an OCT scan. Crown models without roots and tooth models with roots are registered at time T₀.

In the process shown in FIGS. 15 and 16, the tooth arch form optimization procedure described previously is applied to the data acquired at time T₀, producing a first set of motion vectors V₁. These initial movement vectors can be considered as “scaled” or “scaled-back” versions of the full-scale movement vectors V described with reference to FIG. 14, for example,

V₁=0.5V.

These scaled vectors provide data for a single stage in the treatment procedure. With respect to the arch mapping graph of FIG. 15, for example, vector V₁ provides the indicated movement of the corresponding tooth inertial center from time T₀ to time T₁.

Using this multi-stage sequence, the iterative logic repeats its processing at the end of the first stage, effectively using the second positional data of time T₁ as a starting point, so that the T₁ position replaces the T₀ position and processing continues.

In practice, at time Tₖ where k>0, the 3D crown models without roots can be acquired by using an intraoral optical scanner or an OCT scanner without acquiring another CBCT scan to reduce patient X-ray exposure. Tooth models with roots obtained at time T₀ can be aligned with crown models without roots at time Tₖ so a new set of tooth models with roots that are aligned with crown models without roots is formed at time Tₖ. This new set of tooth models with roots can be used to assess the treatment performance and a new treatment plan can be designed and a set of new tooth movement vectors can be computed.

Through a follow-up AI-inverse operation, the system of the present disclosure produces another second positional digital data of patient dentition based on the new first positional digital data computed at time T₁. As shown in FIG. 16, optimization of the calculated patient dentition yields a further improved arch form, using motion along vector V₂ to produce the new positions at time T₂. Following orthodontic treatment, the resultant arch form provides an improved fit of the dentition components (teeth) for aesthetic and functional requirements.

The exemplary two-stage process (T₀-T₁, T₁-T₂) described with reference to FIGS. 15 and 16 can be generalized as a multi-stage process represented by Tᵢ−Tᵢ₊₁. The multi-stage process can be terminated, in general, at the time Tn when the difference between the first positional digital data and the second positional data diminishes. Or, the completion of the multi-stage adjustment process can be determined using a predefined number of iterations, for example, or by evaluating further movement distance according to a predetermined threshold value.

As shown in the example of FIGS. 14, 15, and 16, the system of the present disclosure can automatically, at time Tᵢ (where i=0,1,2,3, . . . n−1) determined by the orthodontic strategy, decompose the displacement data of the second positional data along tangential and normal directions with respect to the resultant arch form at time Tᵢ.

The process of the present disclosure can automatically determine and/or fabricate, at time Tᵢ, a positional corrective device for the dentition components based on the decomposed displacement data and vector V. If the decomposed displacement data is predominantly tangential, a brace may be preferred. If the dominant component of the decomposed displacement data is normal, an aligner may be preferred. In many cases, a combined aligner and brace device is preferred. Vectors V for each step in the process can be reported, such as displayed, printed, or stored, as well as provided to an appliance design system that provides fabrication of a suitable brace, aligner, or other dental appliance for tooth re-positioning, as described in more detail subsequently.

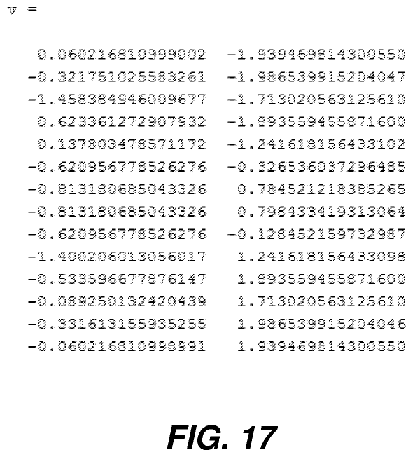

By way of example, FIG. 17 shows a computer-generated listing of movement vectors V (translational elements only for this example) for each of 14 teeth of a dental arch for an orthodontic patient. Values for x- and y-axis movement are shown for each tooth vector V. Movement vector V values can be provided as a listing, such as in a file, or displayed to the practitioner.

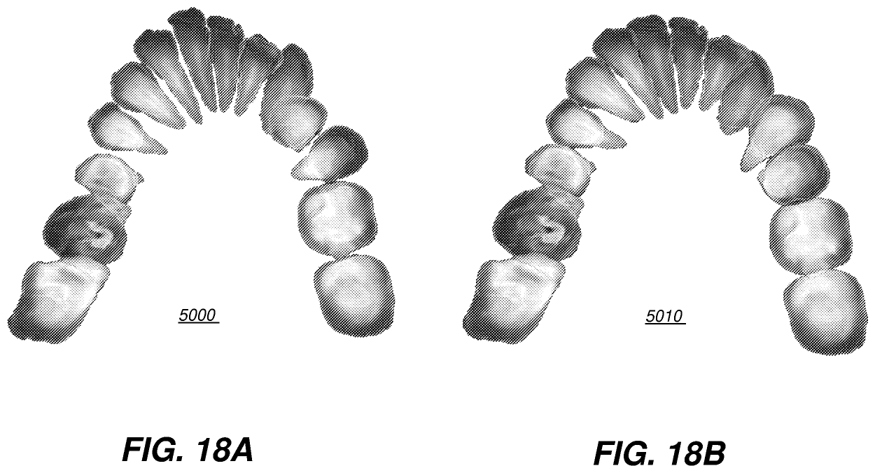

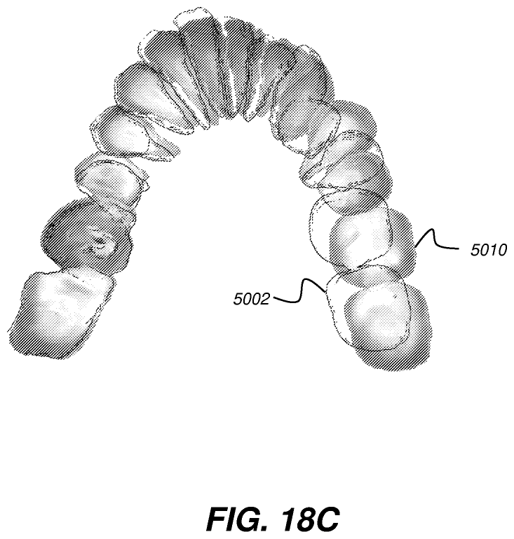

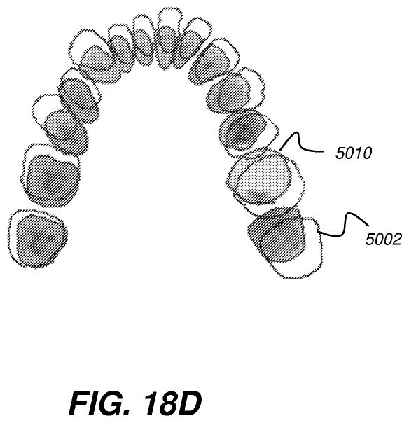

FIG. 18A (a 3D rendering view) shows the arrangement of teeth in an initial, uncorrected arch 5000 for an exemplary orthodontic patient. FIG. 18B shows the corrected arch 5010 following treatment recommendations provided by the system of the present disclosure. FIG. 18C (also partially a 3D rendering view) shows the initial and optimized arches overlaid, with the original tooth positions indicated in outline 5002. FIG. 18D (a slice of a 2D axial view) shows another example with the initial arch in outline 5002, overlaid on the optimized arch 5010.

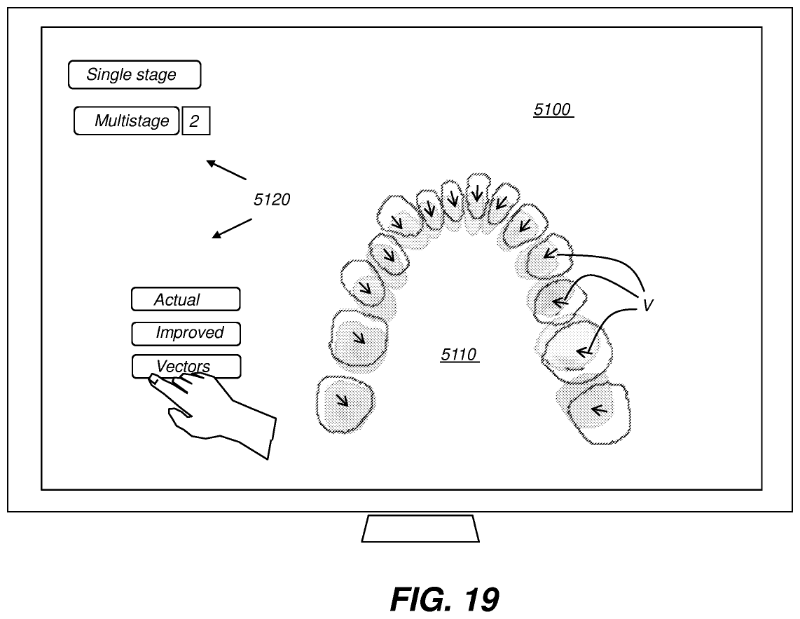

By way of example, the schematic view of FIG. 19 shows an operator interface display 5100 for controlling the processing to provide orthodontic data and for display of processing results. An image portion 5110 provides a graphic representation of actual scanned results (as in FIG. 18A), improved positioning (as in FIG. 18B), and vectors V for proposed tooth movement (as in FIG. 14). These different displayed data can be overlaid, for example, or shown separately as selected by the operator using controls 5120. The operator can also select different calculations and results depending on variable selections, such as for single stage or multi-stage treatment, as described with reference to FIGS. 15 and 16.

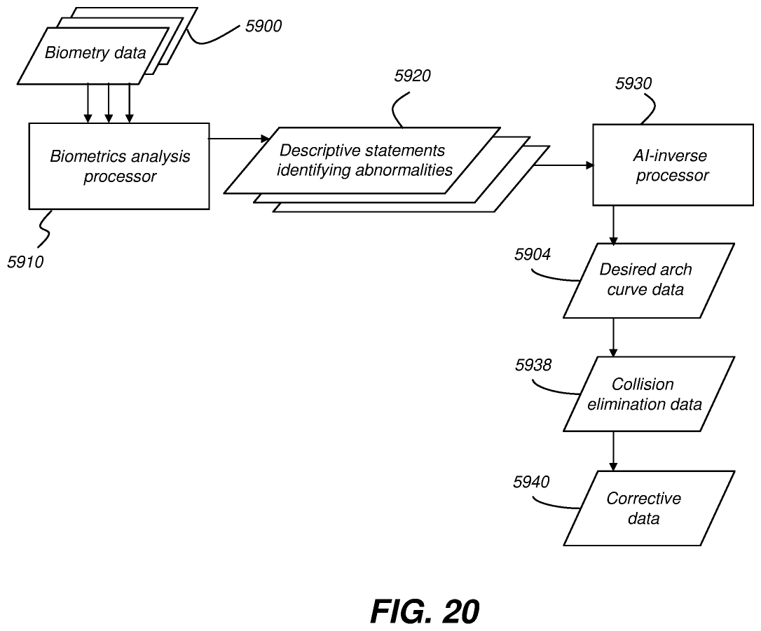

The flow diagram of FIG. 20 shows the logic processing mechanisms and data that can be used for providing assessment and guidance to support orthodontic applications. Comparing to the process illustrated in FIG. 7, the process in FIG. 20 further includes steps for eliminating potential collision that could happen in orthodontic treatment. Biometry data 5900, obtained from CBCT, optical scanning, OCT or other source, and population data is input to a biometrics analysis processor 5910. In response, biometrics analysis processor 5910, an artificial intelligence (AI) engine, performs calculations as described previously and generates descriptive statements 5920 identifying one or more dental/maxillofacial abnormalities.

According to an embodiment of the present disclosure, descriptive statements describing one or more dental/maxillofacial abnormalities such as teeth misalignment, which activates arch shape optimization process to produce teeth motion vectors that in turn to generate a teeth position and orientation rearranged digital model for appliance fabrication.

Continuing with the FIG. 20 process, an AI-inverse processor 5930 provides a logic engine. This AI-inverse engine generates recommended or desired arch curve data 5904, based on and generated from anatomy of the individual patient.

Then, based on the descriptive statements 5920 and the calculated desired arch curve data 5904, a corrective data (similar with data 3940) can be calculated, i.e. a set of potential new positions of all individual tooth can be predicted with calculation. According to these new positions, potential collision is detected. From these predicted collision, a collision elimination data 5938, i.e. a displacement data for eliminating the detected collision is derived.

Thus, for calculating desired movement vectors for individual teeth within the dental arch, both of the desired arch curve data 5904 and collision elimination data 5938 can be considered. According to an embodiment of the present disclosure, the displacement data from desired arch curve data 5904 and displacement data from collision elimination data 5938 are combined, to provide corresponding corrective data 5940 for repositioning the one or more teeth in the dental arch.

Overall, based on the descriptive statements 5920, the desired arch curve data 5904, and the collision elimination data 5938, AI-inverse processor 5930 generates corresponding corrective data 5940. The corrective data 5940 can include motion vectors needed for tooth repositioning, and related data for guidance in orthodontics.

According to an embodiment of the present disclosure, the arch shape optimization process uses teeth inertia centers as the input.

According to another embodiment of the present disclosure, the arch shape optimization process uses combination of segmented cortical bone shape and teeth inertia centers as the input.

According to an embodiment of the present disclosure, collision elimination is performed in a step of virtual set up before fabricating orthodontic treatment appliances, which practically eliminates the need of interproximal reduction procedure that is conventionally adopted by orthodontic practitioners.

According to another embodiment of the present disclosure, the said collision elimination could be performed after the fabrication of the appliance by employing the trimming of the effected teeth if the collision is miniscule and the trimming is guided by the computed collision elimination motion vector.

FIG. 21A is a diagram showing a distribution of teeth in an initial uncorrected dental arch from an exemplary orthodontic patient. FIG. 21B shows an aligned dental arch of the original showing in FIG. 21A based on corrected data, such as corrected data 3940 illustrated in FIG. 7. FIG. 21B indicates that the dental arch shape is optimized, and teeth misalignment has been corrected from the original arch in FIG. 21A, according to a treatment guidance corresponding to FIG. 7 for example. Teeth collision happens, as indicated by green regions in FIG. 21B.

FIG. 22 is a logic flow diagram showing process to calculate a moving direction and a moving distance of a tooth digital model with collisions according to an embodiment of the present invention.

In Step S610, a unique code value is assigned to every individual tooth digital model, i.e. a tooth volume, so different code values are assigned between two or more teeth volumes of the dental arch. For example, each voxel of tooth T1 is assigned with a code value C1, each voxel of tooth T2 is assigned with a code value C2, each voxel of tooth T3 is assigned with a code value C3.

After arch optimization by rearranging the teeth, these code values will appear in different locations on the CBCT head volume from the locations before the arch optimization. Sometimes, two different codes values appear in the same locations where collision occurs.

Therefore, in Step S620, a search in 2D or 3D space of the CBCT head volume is carried on to find collision (engaged) subvolumes with two different code values.

In Step S630, it makes teeth volumes (Tₖ and Tₖ₊₁) associated with subvolume C_{k,k+1} as teeth with collisions.

In Step S640, a tangential vector, TV_{k, k+1} is computed, with respect to the polycurve (an optimal polycurve), such as a 4th order polycurve shown in FIG. 12, using the center location of collision subvolume C_{k,k+1}. An exemplary process of computing the tangential vector TV_{k,k+1} is as follows. Perpendicularly projecting the center location C_{k,k-1} of collision subvolume to the polycurve to obtain a point P. Compute a tangential vector T at P along the polycurve. The direction of tangential vector TV_{k,k+1} will be the same as T but at location of C_{k,k+1}.

In Step S650, a search of the subvolume C_{k,k+1} along a certain direction, such as the tangential vector TV_{k, k+1}, is carried out to find the maximum collision value, i.e. volume thickness d_{k, k+1}. The volume thickness can be used for calculating a compensation of the collision displacement. According to an embodiment of the present disclosure, the compensation is a value equal to the collision volume thickness. According to another embodiment of the present disclosure, the compensation is a value of the collision volume thickness plus a value of tolerance. The tolerance can be a fixed value based on usual experience, or a variable related to another issue, such as the volume of a tooth and the like.

In Step S660, the tooth volume Tₖ or Tₖ₊₁ is moved by an amount of d_{k,k+1} along a direction, such as the direction of the tangential vector −TV_{k, k+1} or TV_{k,k+1}, so that tooth volumes Tₖ and Tₖ₋₁ become disengaged, which means collision between tooth volumes Tₖ and Tₖ₋₁ is eliminated. One of the movement options in Step S660 is shown in FIG. 21D, which is a diagram indicating a moving direction and a moving distance of a tooth digital model according to an embodiment of the present invention.

When potential displacements related to collision are calculated and considered, extra guidance information can be added for orthodontic treatment. In an embodiment of the present disclosure, the displacement data from desired arch curve data and displacement data from collision elimination data are combined. In an embodiment of the present disclosure, the combination is simply an addition of vectors corresponding to displacement data from desired arch curve data and collision elimination data, which is familiar to the people skilled in the art.

FIG. 21C is a diagram showing a digitally optimized arch shape without teeth collision. This is an illustration of an exemplary result of an orthodontic treatment with the consideration of teeth collision by rearranging teeth positions and orientations using said combination motion vector described above.

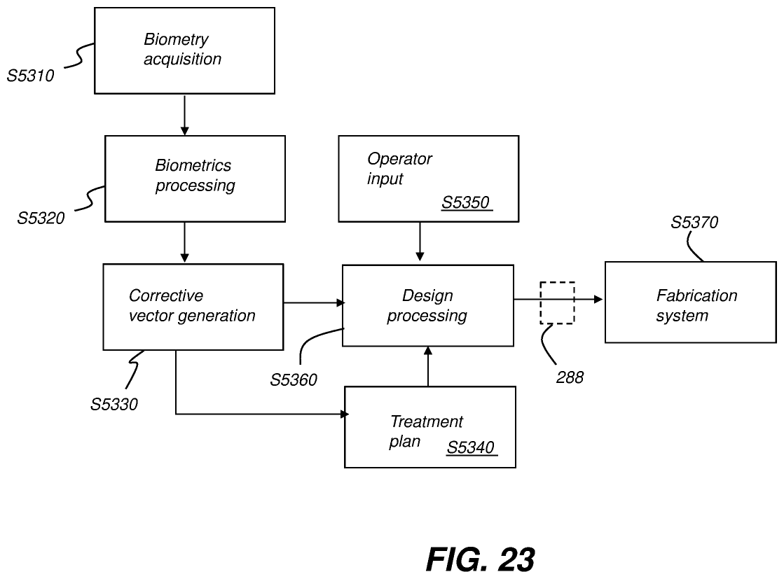

The flow diagram of FIG. 23 shows a sequence for applying results of the orthodontic optimization to the task of appliance design and fabrication. A biometry acquisition step S5310 obtains the biometry data described hereinabove to characterize the dental geometry of interest for orthodontic treatment. A biometrics processing step S5320 then performs the processing of the dental data, generating the type of data describing one or more dental/maxillofacial abnormalities. A vector generation step S5330 then generates corrective data that dictates desired movement vectors for individual teeth, as was described with reference to FIG. 19 and FIG. 21. The corrective data can be generated using the AI-inverse processor described previously, for example. According to an embodiment of the present disclosure, the corrective data is calculated with consideration of desired arch curve data, as described with FIG. 7. According to another embodiment of the present disclosure, the corrective data is calculated with consideration of both desired arch curve data and collision elimination data, as described with FIG. 20. This corrective information can be integrated with a treatment plan in a treatment plan generation step S5340. An operator input entry step S5350 can provide additional data that is used to support a design processing step S5360, which can be fully automated partially automated, or predominantly manual. The output of design processing step S5360 goes to a fabrication step S5370, executed by a fabrication system, such as a 3D printer or other device for appliance manufacture.

According to an embodiment of the present disclosure, design processing step S5360 translates the movement vector data generated automatically for the treatment plan into design data that supports the automated fabrication of a suitable dental appliance. As its output, design processing step S5360 can generate a suitable file for 3D printing, such as a .STL (Standard Triangulation Language) file, commonly used with 3D printers, or a .OBJ file that represents 3D geometry. Other types of print file data can be in proprietary format, such as X3G or FBX format.

In fabrication step S5370, automated fabrication systems can be additive, such as 3D printing apparatus that uses stereolithography (SLA) or other additive method that generates an object or form by depositing small amounts of material onto a base structure. Some alternate methods for additive fabrication include fused deposition modeling that applies material in a liquid state and allows the material to harden and selective laser sintering, using a focused radiant energy to sinter metal, ceramic, or polymer particulates for forming a structure. Alternately, automated fabrication devices can be subtractive, such as using a computerized numerical control (CNC) device for machining an appliance from a block of a suitable material.

User interaction can be employed as part of the fabrication process, such as to verify and confirm results generated and displayed automatically, or to modify generated results at practitioner discretion. Thus, for example, the operator can accept some guidance from the automated system, but alter the generated movement data according to particular patient needs.

The present invention provides a method and a system for fabricating a dental appliance for orthodontic treatment, where an improved teeth digital model for fabricating physical aligners is provided according to incremental positions and orientation and corresponding movement vectors for individual teeth within the dental arch, which are acquired from optimal arch forms for the patient's dental arch. The corresponding movement vectors include translational and angular elements for position and orientation correction. Specific details for generating the optimal arch forms for the patient's dental arch have been introduced in the previous text. The fabricating methods and system of the present invention includes determination of a digital model of an orthodontic aligner with additive device based on the incremental positions and movement vectors. Comparing with most of the conventional aligner, the new aligner introduced in the present invention provides more specialized forces to the individual teeth, so as to improve the efficiency in desired orthodontic treatment.

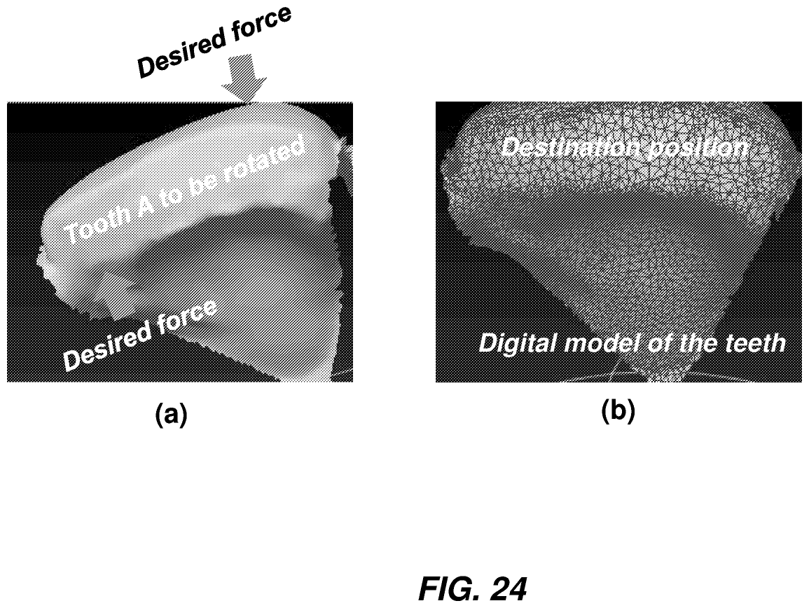

FIG. 24 is a diagram showing an example of tooth repositioning. FIG. 24 (a) illustrates an original position of a tooth within the dental arch, which need to be rotated according to the destination position as shown in FIG. 24 (b) calculated by the digital model of the teeth based on methods described previously. For the tooth repositioning shown in FIG. 24 for example, desired forces illustrated with arrows in FIG. 24 (a) need to be applied to the tooth.

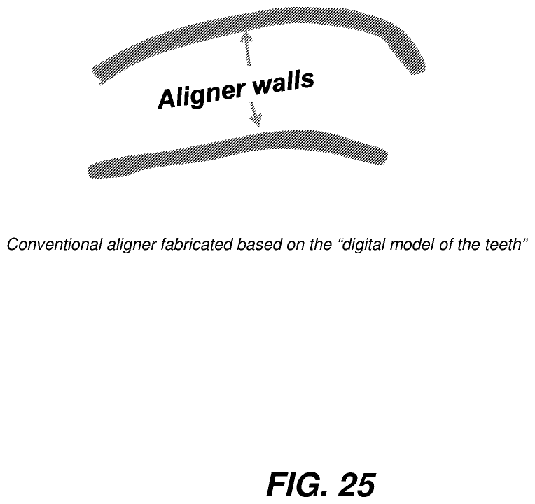

FIG. 25 is a diagram showing an example of a portion of a conventional aligner fabricated with exemplary thermoplastic materials based on the digital model of the teeth by considering the desired destination positions. Each aligner is designed for an incremental position adjustment over the teeth. As shown in FIG. 26, a diagram showing an example of a conventional aligner on the teeth to be repositioned, the intended forces for the tooth rotational movement as indicated with arrows are provided by the aligner over the patient's teeth.

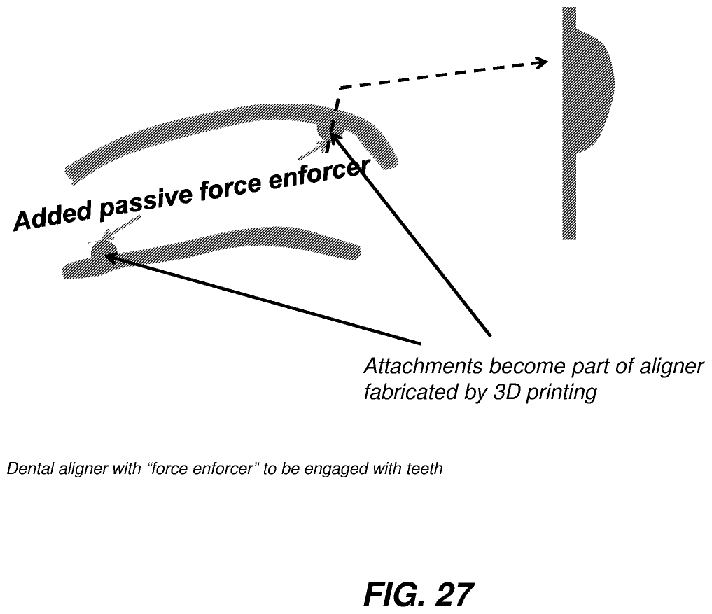

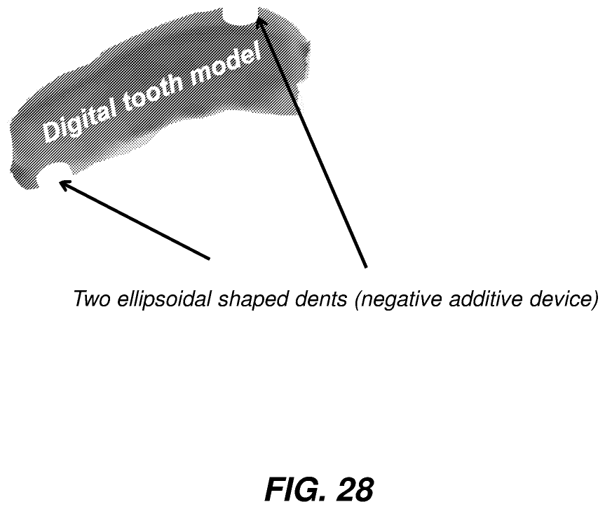

FIG. 27 is a diagram showing an example of a modified aligner fabricated with additive devices (passive force enforcer) based on the digital model determined according to an embodiment of the present invention. Transparent orthodontic aligners are made from thermoplastic materials, for example. Using thermoforming machine to fabricate an aligner, a physical model is placed in a thermoforming caster, and heat and vacuum were applied during thermoforming. FIG. 28 depicts part of a digital model corresponding to the model, with two negative ellipsoids added to produce two active force enforcers on the inner surface of the aligner.

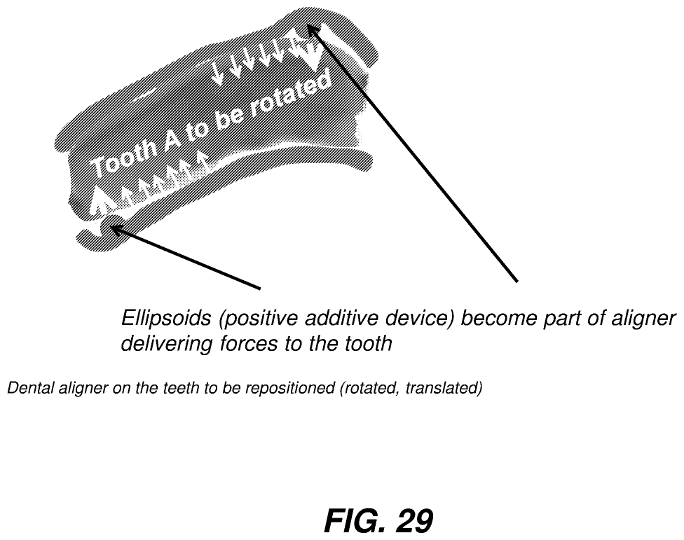

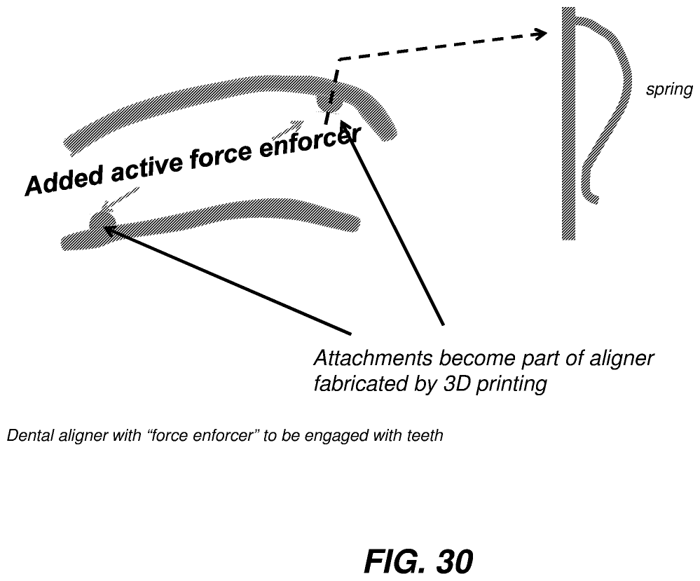

FIG. 29 is a diagram showing an example of a modified aligner fabricated based on the digital model determined in the present invention on the tooth to be repositioned. The added passive force enforcer causes additional deformation of the aligner, increasing the pressure in the direction of the intended forces. Thus, the added passive force enforcer enhances the effectiveness of the orthodontic treatment process, and even shortens the treatment cycle. FIG. 30 is a diagram showing an operating principle of a modified aligner with an active force enforcer (a spring like attachment on the inner surface of the aligner) according to an embodiment of the present invention. The exemplary shape of the enforcer could be ellipsoidal or rectangular among other possible shape designs that are effective for the desired tooth movement. An exemplary dimension of an ellipsoid device could be 3 mm in height, 2 mm in width and 1 mm in depth. An exemplary dimension of a rectangular device could be 5 mm in height, 2 mm in width and 1 mm in depth. For each of the intermediate teeth models, carefully selected digital enforcers are added to the proper locations on the surface of the teeth that need to be moved or rotated. For example, in FIG. 29, there are two exemplary passive ellipsoid devices (called positive ellipsoids) are attached to the aligner in order to give an extra push to the tooth at the right locations for a rotation correction.

These two ellipsoid devices are digitally added to the digital tooth model at the locations indicated in FIG. 28 in such a way that there will be two ellipsoidal shaped dents (also called negative ellipsoids) on the tooth surface.

This enforcer device added intermediate teeth model or the final teeth model is used in a 3D printer to produce a solid intermediate physical teeth model, i.e. the physical model. Using this solid intermediate or final physical teeth model with one or more negative additive devices (such as the exemplary two dents) on its surface, an aligner on its inner wall having positive additive devices (such as two positive ellipsoidal enforcers) can be readily fabricated using exemplary thermoplastic materials in an exemplary a thermoforming machine, which is well known to people skilled in the art.

It should be understood by those skilled in the art that the size, shape, position, and number of the added passive force enforcers shown in the drawings are for illustrative purposes only, and not intended as limitation of the scope of the present invention.

Similar with the fabrication of the conventional aligner, the improved digital model for fabricating physical aligners with added passive force enforcers can be designed fully by the computer program with corresponding algorithms of aligner designs for orthodontic treatment. However, as illustrated in step S5350 of FIG. 23, the operator can provide additional data and input that is used to support a design processing step S5360. The operator input could be based on the practice experience or patient's need or preference.

Described herein is a computer-executed method and system to provide support and guidance for subsequent treatment, including fabrication of appropriate dental appliances using manual or automated methods.

Consistent with exemplary embodiments herein, a computer program can use stored instructions that perform 3D biometric analysis on image data that is accessed from an electronic memory. As can be appreciated by those skilled in the image processing arts, a computer program for operating the imaging system and probe and acquiring image data in exemplary embodiments of the application can be utilized by a suitable, general-purpose computer system operating as control logic processors as described herein, such as a personal computer or workstation. However, many other types of computer systems can be used to execute the computer program of the present invention, including an arrangement of networked processors, for example. The computer program for performing exemplary method embodiments may be stored in a computer readable storage medium. This medium may include, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. Computer programs for performing exemplary method embodiments may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the application, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer is also considered to be a type of memory, as the term is used in the application. Memory is also used as the data workspace for executing and storing intermediate and final results of calculations and other processing. Computer-accessible memory can be volatile, non-volatile, or a hybrid combination of volatile and non-volatile types.

It will be understood that computer program products of the application may make use of various image manipulation algorithms and processes that are well known. Additional aspects of such algorithms and systems, and hardware and/or software for producing and otherwise processing the images or co-operating with the computer program product exemplary embodiments of the application, are not specifically shown or described herein and may be selected from such algorithms, systems, hardware, components and elements known in the art.

While the invention has been illustrated with respect to one or more implementations, alterations and/or modifications can be made to the illustrated examples without departing from the spirit and scope of the appended claims. In addition, while a particular feature of the invention can have been disclosed with respect to only one of several implementations/embodiments, such feature can be combined with one or more other features of the other implementations/embodiments as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. Other embodiments of the invention will be apparent to those skilled in the art from consideration of the specification and practice of the invention disclosed herein. It is intended that the specification and examples be considered as exemplary only, with a true scope and spirit of the invention being indicated by at least the following claims.

1. A method for fabricating a dental appliance for orthodontic treatment, at least partially performed by a computer, the method comprising the steps of:
(a) obtaining three-dimensional dental data from a patient's scan, including at least one dental arch of the patient;
(b) locating initial tooth positions along the dental arch from the three-dimensional dental data;
(c) generating optimal arch forms for the patient's dental arch to acquire incremental positions and corresponding movement vectors for individual teeth in the dental arch;
(d) determining a digital model for fabricating an orthodontic aligner with an additive device based on the incremental positions and movement vectors;
(e) displaying, storing, or transmitting the determined digital model.

2. The method of claim 1, further comprising the steps of:
(f) producing a physical model according to the determined digital model using a 3D printer;
(g) fabricating a physical aligner with the additive device using the physical model.

3. The method of claim 1, wherein the three-dimensional dental data includes a three-dimensional volume representing the dental anatomy of a patient acquired using a cone beam computed tomography system.

4. The method of claim 1, wherein the three-dimensional dental data includes three-dimensional surfaces representing a tooth or teeth of a patient acquired using an intraoral optical scanner.

5. The method of claim 1, wherein the three-dimensional dental data is acquired using an optical coherence tomography (OCT) system.

6. The method of claim 1, wherein the movement vectors are provided as a listing of coordinate values and/or angles.

7. The method of claim 1, wherein the step of generating optimal arch forms for the patient's dental arch comprises the steps of:
(a) selecting first positional digital data for one or more teeth from the located initial tooth positions along the dental arch from the three-dimensional dental data;
(b) generating second positional digital data for the one or more teeth according to a desired dental arch form for the patient;
(c) calculating displacement data for one or more teeth according to the first positional and second positional digital data; and
(d) calculating an intermediate displacement for incremental positions and corresponding movement vectors for the one or more teeth.

8. The method of claim 1, wherein the step of generating optimal arch forms for the patient's dental arch comprises steps of:
(a) selecting first positional digital data for one or more teeth from the located initial tooth positions along the dental arch from the three-dimensional dental data;
(b) generating second positional digital data for the one or more teeth according to a desired dental arch form for the patient;
(c) calculating first displacement data for one or more teeth according to the first positional and second positional digital data;
(d) detecting teeth collision values based on the first displacement data;
(e) calculating second displacement data for one or more teeth based on the detected teeth collision values;
(f) combining the first displacement data and second displacement data;
(g) calculating an intermediate displacement for incremental positions and corresponding movement vectors for the one or more teeth; and
(h) reporting the intermediate displacement for repositioning one tooth or more teeth of the dental arch.

9. The method of claim 8, wherein the step of detecting teeth collision values comprises the steps of:
(a) assigning separate code values to two or more teeth volumes;
(b) searching in 2D or 3D space to find a collision subvolume of two teeth volumes with the code values;
(c) marking teeth volumes associated with the collision subvolume as teeth volumes with collision.

10. The method of claim 8, wherein the step of calculating second displacement data comprises the steps of:
(a) deciding a directional value of the collision subvolume;
(b) searching the subvolume along a direction corresponding to the decided directional value to find a maximum collision value;
(c) computing second displacement data based on the maximum collision value.

11. The method of claim 8, wherein the step of combining first displacement data and second displacement data comprises an addition of vectors corresponding to the first displacement data and second displacement data.

12. The method of claim 1, wherein the position of an individual tooth is inertia center of the teeth.

13. A system for dental orthodontic treatment, the system comprising:
(a) a scanning apparatus configured to acquire three-dimensional dental data from a scan of a patient's teeth;
(b) a computer apparatus programmed with instructions for:
(i) locating initial tooth positions along a dental arch from the three-dimensional dental data;
(ii) generating optimal arch forms for the patient's dental arch to acquire incremental positions and corresponding movement vectors for individual tooth in the dental arch;
(iii) determining a digital model for fabricating an orthodontic aligner with an additive device based on the incremental positions and movement vectors;
(iv) displaying, storing, or transmitting the determined digital model.

14. The system of claim 13, wherein the scanning apparatus includes: (i) a cone beam computed tomography (CBCT) system, (ii) an intraoral optical scanner, (iii) an optical coherence tomography (OCT) system, or (iv) any combination of the foregoing.

15. The system of claim 13, wherein the system further comprises:
(a) a 3D printer for producing a physical model according to the determined digital model, wherein the 3D printer is in signal communication with the computer apparatus; and
(b) an apparatus for fabricating a physical aligner with additive device using the physical model.

16. A method for fabricating a dental appliance for orthodontic treatment executed at least in part by a computer, the method comprising the steps of:
(a) acquiring three-dimensional data from scans of maxillofacial and dental anatomy of a patient;
(b) computing a plurality of cephalometric values from the acquired three-dimensional data;
(c) processing the computed cephalometric values and generating metrics indicative of tooth orientation and tooth positioning along a dental arch of the patient;
(d) analyzing the generated metrics to calculate desired movement vectors for individual teeth within the dental arch;
(e) determining a digital model of intermediate or final teeth arrangement based on the desired movement vectors;
(f) determining a digital model of additive devices to support teeth movements corresponding to the determined digital model of teeth arrangement;
(g) displaying, storing, or transmitting the digital model of teeth arrangement and the digital model of additive devices;
(h) producing physical teeth models with negative physical additive devices by performing 3D printing using the digital models of teeth arrangement and digital model of additive devices; and
(i) fabricating a physical aligner with positive additive devices using the physical teeth model.
